(19) United States
(12) Patent Application Publication
(10) Pub. No.: US 2021/0217508 A1
(43) Pub. Date: Jul. 15, 2021

(54) Graded Exercise Therapy Systems and Methods

(71) Applicants: ANSwers Neuroscience Inc., New York, NY (US); Cornell University, Ithaca, NY (US)

(72) Inventors: Barry Kosofsky, New York, NY (US); Phil Celmer, Flemington, NJ (US); Juhani Perhonen, Hamden, CT (US)

(21) Appl. No.: 16/896,927

(22) Filed: Jun. 9, 2020

Related U.S. Application Data
(60) Provisional application No. 62/959,562, filed on Jan. 10, 2020.

(51) Int. Cl. G16H 20/30; G16H 10/60; G16H 50/30; G16H 40/67; G16H 10/40; G16H 10/20; G16H 20/70; A63B 24/00; A61H 33/00; G01K 13/00

(52) U.S. Cl. CPC G16H 20/30; A61H 2201/5082; G16H 50/30; G16H 40/67; G16H 10/40; G16H 10/20; G16H 20/70; A63B 24/0075; A61H 33/00; G01K 13/00; A63B 2024/0068; A63B 2230/06; A63B 2230/30; A63B 2230/207; A63B 2208/03; G16H 10/60

(57) ABSTRACT

Various embodiments provide novel tools and techniques for providing graded exercise therapy. A system includes one or more sensors coupled to a patient, and a host machine coupled to the one or more sensors. The host machine includes a processor, and a computer readable medium in communication with the processor having encoded thereon a set of instructions executable by the processor to establish a graded exercise therapy regime for the patient, determine one or more physiologic data targets for the patient, obtain a first set of physiologic data of the patient during the graded exercise therapy regime, and update the one or more physiologic data targets based, at least in part, on the first set of physiologic data.

### CROSS REFERENCE TO RELATED APPLICATIONS

This application claims priority to U.S. Provisional Patent Application Ser. No. 62/959,562, filed Jan. 10, 2020 by Barry E. Kosofsky (attorney docket no. 8237-01-US), entitled “Graded Exercise Therapy Systems and Methods,” the entire disclosure of which is incorporated herein by reference in its entirety for all purposes.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### FIELD

The present disclosure relates, in general, to treatment of autonomic dysfunction, and more particularly, to novel tools and techniques for providing graded exercise therapy in the treatment of autonomic dysfunction.

### BACKGROUND

Concussion is a brain injury that may produce various symptoms that are difficult to evaluate and treat, often relying on a physician's subjective assessment of a patient. One such approach to treating persistent post-concussion symptoms (PPCS) is the Buffalo Concussion Treadmill Test (BCTT). The BCTT identifies individuals exhibiting autonomic nervous system (ANS) dysfunction that significantly contributes to more prolonged PPCS. Another similar test includes the Buffalo Concussion Bicycle Test (BCBT). The basis for ANS dysfunction in contributing to PPCS presumably lies in a defect in cerebral auto-regulation. Following a concussion, impaired auto-regulation results in blood flow to the brain not being appropriately coupled to brain or body activity, resulting in effort-induced headache, the hallmark of PPCS. Such individuals usually become symptomatic with headache and an inability to exercise further when their heart rate rises above approximately the 130 beat per minute (bpm) mark.

Graded exercise therapy (GET) has been used to accelerate ANS recovery, coordinate with greater tolerance for physical and mental activities following concussion. However, GET is typically provided under the guidance of a clinician (or other care provider), and results of the GET rely on a subjective evaluation or manually measurement by the clinician. Moreover, GET has typically been limited to treating athletes following concussion. A broader array of chronic diseases of “deconditioning,” as well as degenerative neurologic disorders and diseases at the intersection of brain and heart health often, though not always, associated with ANS dysfunction, are not treated through GET.

Accordingly, tools and techniques for providing graded exercise therapy for a broader range of disorders of brain and heart health are provided.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. In some instances, a sub-label is associated with a reference numeral to denote one of multiple similar components. When reference is made to a reference numeral without specification to an existing sub-label, it is intended to refer to all such multiple similar components.

FIG. 1 is a schematic block diagram of a system for providing graded exercise therapy, in accordance with various embodiments.

FIG. 2 is a functional block diagram of a graded exercise therapy system, in accordance with various embodiments.

FIG. 3 is a hardware block diagram of a system for providing graded exercise therapy, in accordance with various embodiments.

FIG. 4 is a hardware block diagram of an alternative system for providing graded exercise therapy, in accordance with various embodiments.

FIG. 5 is a flow diagram of a method for providing graded exercise therapy, in accordance with various embodiments.

FIG. 6 is a schematic block diagram of a computer system for providing graded exercise therapy, in accordance with various embodiments.

FIG. 7 is a schematic block diagram illustrating system of networked computer devices.

### DETAILED DESCRIPTION OF CERTAIN EMBODIMENTS

While various aspects and features of certain embodiments have been summarized above, the following detailed description illustrates a few exemplary embodiments in further detail to enable one of skill in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments may be practiced without some of these specific details. In other instances, certain structures and devices are shown in block diagram form. Several embodiments are described herein, and while various features are ascribed to different embodiments, it should be appreciated that the features described with respect to one embodiment may be incorporated with other embodiments as well. By the same token, however, no single feature or features of any described embodiment should be considered essential to every embodiment of the invention, as other embodiments of the invention may omit such features.

Unless otherwise indicated, all numbers used herein to express quantities, dimensions, and so forth used should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

In an aspect, a system for providing graded exercise therapy is provided. The system includes one or more sensors coupled to a patient, and a host machine coupled to the one or more sensors. In various embodiments, the host machine may further include a processor, and a computer readable medium in communication with the processor, the computer readable medium having encoded thereon a set of instructions executable by the processor to provide GET. For example, host machine may execute the instructions to determine, via the one or more sensors, a baseline set of physiologic data of the patient. The host machine may further be configured to establish a graded exercise therapy regime for the patient, the graded exercise therapy regime including one or more exercises, and determine one or more physiologic data targets for the patient based, at least in part, on the baseline physiologic data of the patient. The host machine may further be configured to obtain, via the one or more sensors, a first set of physiologic data of the patient during the graded exercise therapy regime, determine whether the first set of physiologic data meets the one or more physiologic data targets, and in response to determining that the first set of physiologic data meets the one or more physiologic data targets, update the one or more physiologic data targets based, at least in part, on the first set of physiologic data.

In another aspect, an apparatus for providing graded exercise therapy is provided. The apparatus includes a processor, and a computer readable medium in communication with the processor, the computer readable medium having encoded thereon a set of instructions executable by the processor to provide GET. For example, the instructions may be executed by the processor to determine, via one or more sensors, a baseline set of physiologic data of a patient, establish a graded exercise therapy regime for the patient, the graded exercise therapy regime including one or more exercises, and determine one or more physiologic data targets for the patient based, at least in part, on the baseline physiologic data of the patient. The instructions may further be executed by the processor to obtain, via the one or more sensors, a first set of physiologic data of the patient during the graded exercise therapy regime, determine whether the first set of physiologic data meets the one or more physiologic data targets, and in response to determining that the first set of physiologic data meets the one or more physiologic data targets, update the one or more physiologic data targets based, at least in part, on the first set of physiologic data.

In a further aspect, a method for providing graded exercise therapy is provided. The method includes determining, via one or more sensors, a baseline set of physiologic data of the patient, establishing, via a host computer, a graded exercise therapy regime for the patient, the graded exercise therapy regime including one or more exercises, and determining, via the host computer, one or more physiologic data targets for the patient based, at least in part, on the baseline physiologic data of the patient. The method further includes obtaining, via the one or more sensors, a first set of physiologic data of the patient during the graded exercise therapy regime, and determining, via the host computer, whether the first set of physiologic data meets the one or more physiologic data targets. In response to determining that the first set of physiologic data meets the one or more physiologic data targets, the method may continue by updating, via the host computer, the one or more physiologic data targets based, at least in part, on the first set of physiologic data.

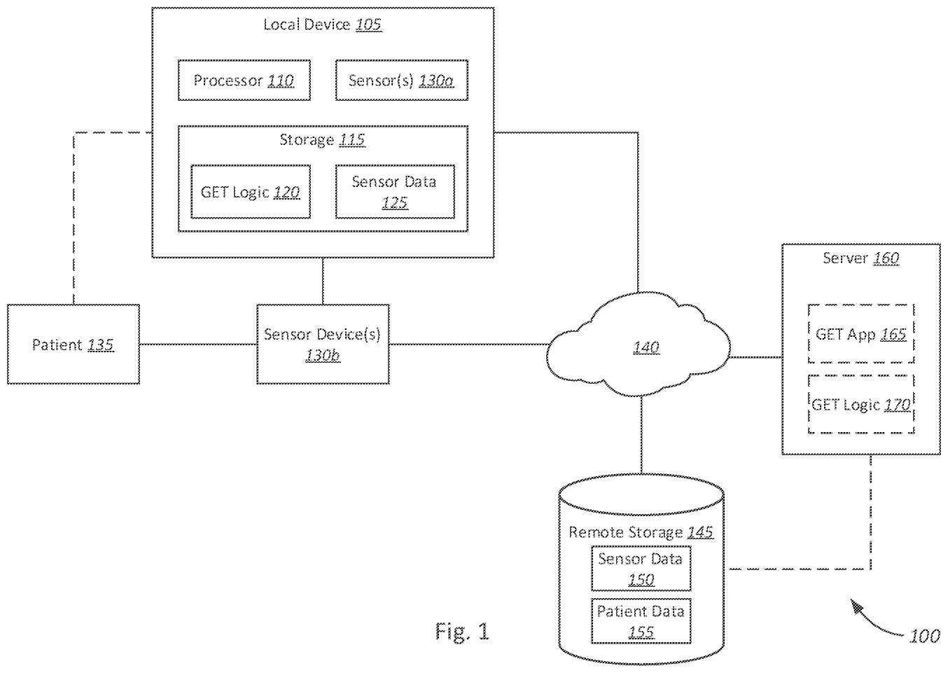

FIG. 1 is a schematic block diagram of a system 100 for providing graded exercise therapy (GET), in accordance with various embodiments. The system 100 includes a local device 105 further including a processor 110, storage 115, and one or more sensors 130a. The storage 115 may further includes GET logic 120, and sensor data 125. The system 100 further includes one or more sensor devices 130b, communication network 140, remote storage 145, which further includes sensor data 150 and patient data 155, and server 160, which included GET app 165 and GET logic 170. It should be noted that the various components of the system 100 are schematically illustrated in FIG. 1, and that modifications to the system 100 may be possible in accordance with various embodiments.

In various embodiments, the local device 105 may include a processor 110, storage 115, and one or more sensors 130a. The storage 115 of the local device 105 may include GET logic 120 and sensor data 125. The local device 105 may be coupled to one or more sensor device 130b, the patient 135, and/or the communication network 140. The one or more sensor devices 130b may further be coupled to the patient 135 and the communication network 140. In some embodiments, the local device 105 may be coupled to one or more of the server 160 and remote storage 145 via the network 140. In some embodiments, the server 160 may further be coupled to the remote storage 145. In further embodiments, the one or more sensor devices 130b may be coupled to the server 160 and/or remote storage 145 via the communication network 140.

In various embodiments, the local device 105 may include a local computer device. For example, the local device 105 may include, without limitation, a mobile device, such as a smartphone, tablet, wearable device, an internet-of-things (IoT) device, or other wireless communication device, a personal computer, desktop computer, workstation, or server computer. In some embodiments, the local device 105 may be configured to provide GET to a patient 135 directly, and/or via the one or more sensor device(s) 130b. Accordingly, in some embodiments, the local device 105 may be configured to obtain, via the communication network 140, a GET app 165 from the server 160. Accordingly, in some embodiments, the GET logic 120 may include GET app 165, which may be executed by the local device 105 to perform one or more commands, as will be described below. In other embodiments, the server 160 may be a remote server accessible, via the communication network 140, which may be configured to execute GET logic 170 and to provide GET to the patient 135 remotely, through the local device and/or one or more sensor devices 130b. In yet further embodiments, the one or more sensor devices 130b, such as a mobile device or wearable device, may be configured to execute at least part of the GET logic and to provide GET to the patient 135.

Accordingly, in various embodiments, the local device 105 may be configured to provide GET to the patient 135. GET may include obtaining one or more physiologic data from the patient 135 (e.g., a set of physiologic data), which may be stored as sensor data 125. For example, physiologic data may include, without limitation, heart rate (HR), heart rate variability (HRV), blood pressure (BP), beat to beat blood pressure (b-bBP) and blood pressure variability (BPV), performance on the cold pressor (e.g., “ice bucket”) test, performance on the BCTT, performance on the BCBT, and/or performance on an appendage or face cooling test, as known to those skilled in the art. In some embodiments, physiologic data may include both raw sensor data 125 and sensor data that has been processed (e.g., HRV, BPV, performance on tests, etc.). In some embodiments, performance on BCTT and BCBT may include physiologic data such as determinations of HRV, BPV, maximum HR, and time to return to resting HR. In some embodiments, performance on the ice bucket test and appendage cooling test may include physiologic data such as appendage temperature, skin temperature, time to return to baseline skin temperature, etc. Accordingly, in various embodiments, one or more tests may be conducted to determine baseline physiologic data (e.g., a baseline set of physiologic data) for GET.

In various embodiments, the local device 105 may be configured to obtain the physiologic data collected by one or more sensors 130a, which may include on-board sensors of a local device 105, such as, without limitation, a camera or other image sensor, heart rate monitor, pulse oximeter, gyroscope, accelerometer, thermometer or other thermal sensor, wireless transceivers, microphone, speaker, or acoustic transceiver, and/or the like. The local device 105 may further be configured to obtain physiologic data collected by the one or more sensor devices 130b, which may be external to the local device 105. For example, the one or more sensor devices 130b may include a mobile device, wearable device, health monitors, etc., which may include one or more sensors, such as a camera or other image sensor, heart rate monitor, pulse oximeter, gyroscope, accelerometer, wireless transceivers, thermometers or other thermal sensors, microphone, speaker, or acoustic transceiver, or the like.

Accordingly, in some embodiments, the local device 105 and/or one or more sensor devices 130b may be coupled to the patient 135. The patient 135 may generate one or more physiologic signals (e.g., a pulse, respiration rate, blood oxygenation, etc.). Accordingly, the one or more sensors 130a of the local device 105 and/or one or more sensor devices 130b may be configured to obtain physiologic data from the patient 135. Data generated by the one or more sensors 130a and/or one or more sensor devices 130b may be referred to as sensor data, which may be stored, for example, in storage 115. Thus, the storage 115 may include sensor data 125.

In some further embodiments, sensor data generated by the one or more sensor devices 130b and/or one or more sensors 130a may be provided to remote storage 145. Thus, the remote storage 145, in some embodiments, may include sensor data 150. For example, in some embodiments, the one or more sensor devices 130b may be configured to transmit sensor data to the remote storage 145, via the communication network 140. For example, the one or more sensor devices 130b may include, for example, a Bluetooth, Wi-Fi, infrared, RF, or other wireless transceiver configured to communicate over communication network 140. Accordingly, the one or more sensor devices 130b may be configured to transmit sensor data over the communication network 140. In some embodiments, the one or more sensor devices and/or the one or more sensors 130a may be configured to provide sensor data to the local device, which may store the sensor data 125 in storage 115. The local device 105 may, in turn, be configured to transmit the sensor data 125 to the remote storage 145 via communication network 140. Accordingly, in some embodiments, sensor data 150 may be obtained by the local device 105 and/or server 160 from remote storage 145. For example, in one arrangement, the local device 105 may obtain, via network 140, the sensor data 150 from the remote server 145.

The local device 105 may accordingly, in some examples, be a host device to which a wearable device, mobile device, and/or remote system may be coupled. The local device 105 may, in turn, be configured to provide GET via the wearable device, mobile device, and/or remote system. Accordingly, in some embodiments, the local device 105 may be configured to provide one or more instructions for the GET regime through the wearable device or mobile device, such as the one or more sensor devices 130b. For example, in some embodiments, the one or more sensor device 130b, which may include the wearable device and/or mobile device, may provide instructions for the GET regime, via a display device, audio speakers, or other interface through which the one or more sensor device 130b may interact with the patient 135. In other embodiments, the local device 105 may be configured to provide GET regime directly to the patient 135, for example, via a display device, audio speakers, or other interface through which the local device 105 may interact with the patient 135.

In various embodiments, the local device 105 may be configured to determine one or more physiologic data targets. For example, in some embodiments, physiologic data targets may include, without limitation, a target HR, target peak HR, target HRV, target BP, target BPV, target temperature, temperature threshold, or other physiologic data targets as appropriate. In some embodiments, the local device 105 may be configured to determine an initial physiologic data target based, at least in part, on the baseline physiologic data. In some embodiments, initial physiologic data targets may be predetermined, provided by a clinician, or empirically derived. In further embodiments, the one or more physiologic data targets may further be determined based, at least in part, on patient data 155. Patient data 155 may include, for example, patient medical history, historical physiologic data, and collateral data (e.g., past performance on one or more tests, or GET regimes). Examples of collateral data may include, without limitation, responses to evaluations and/or tests (e.g., a post-concussion symptom scale (PCSS), Godin Leisure Time Exercise questionnaire, etc.), performance on diagnostic tests (e.g., exercise tolerance test, visual evoked response (VER) tests, etc.), medical history and conditions (e.g., previous concussions, attention deficit disorder, learning disability, peritraumatic amnesia, loss of consciousness, migraines, anxiety, participation in sports), genetic tests and/or therapies (e.g., genetic screening, SNPs, RNA, microRNA and other gene therapies), and other neurological tests and evaluations (e.g., eye tracking, neck movement, balance, etc.). In various embodiments, patient data 155 may itself be updated according to the most recent available data. For example, in one example, collateral data such as PCSS may be administered and updated daily, while a Godin Leisure Time Exercise questionnaire and exercise tolerance test may be conducted and updated weekly.

Once initial physiologic data targets are determined, the GET logic may determine additional physiologic data targets, subsequent to the initial physiologic data targets. For example, in some embodiments, physiologic data targets may be determined based, at least in part, on physiologic data obtained after completion of one or more GET regime, or other test.

A GET regime may include a prescribed exercise regime. Accordingly, in various embodiments, the GET regime may be obtained, via GET logic 120, 170 and/or the one or more sensor devices 130b, from a trainer or clinician interface as provided by a trainer or clinician. In other embodiments, the GET logic 120, 170 and/or one or more sensor devices 130b may be configured to automatically determine a GET regime based on the baseline physiologic data. Accordingly, the GET regime may include one or more exercise therapies and routines, as known to those skilled in the art. For example, the GET regime may include a BCTT training regime, BCBT training regime, or other prescribed GET regime (e.g., an exercise routine). Alternatively, the GET regime may be any exercise of the patient's 135 choice.

For example, in some embodiments, BCBT may be administered as a GET regime to the patient. Before beginning BCBT, a baseline measure of exercise tolerance of the patient may be assessed. For example, in some embodiments, a baseline for exercise tolerance may be assessed based on one or more of a HR, BP, HRV, BPV, rating of perceived exertion (RPE), self-reported concussion symptom severity scale, body weight, and power output during the exercise. To establish a baseline, patient data may be measured when beginning BCBT. For example, in some embodiments, and exercise test may be administered to the patient. Physiologic data of the patient may be measured when the patient is at rest, before the exercise is performed. The patient may then be asked to perform the exercise, in this example, riding a stationary bicycle. The patient may be asked to rate subjectively a level of exertion and symptom severity before, during, and after the exercise. The patient's physiologic data (e.g., HR, BP, body weight, and power output may then be measured before, during, and after the exercise. The patient's physiologic data may further be compared to expected values for individuals with normal exercise tolerance, adjusting for demographic information and characteristics of the patient.

In some embodiments, an initial exercise may be prescribed by a trainer and/or clinician, with an intensity and/or duration based on the baseline measure of exercise tolerance as described above. As the patient progresses, subsequent exercises may be performed with increasing intensity and/or duration. In some examples, when the patient has completed the exercise routine two days in a row without an increase in symptom severity, the intensity of the exercise routine may be increased. In some embodiments, an increase in the intensity of the exercise routine may be increased in predetermined steps relative to the size of the patient. For example, in some embodiments, an initial exercise may be performed with an intensity of 3.1 metabolic equivalent of task (MET). MET is a measure of a rate of energy expenditure relative to the mass of a person for a given exercise relative to a reference task that expends 3.5 mL of oxygen per kilogram per minute. If the exercise is performed two consecutive days without a reported increase in symptom severity, the patient may move onto a subsequent “stage” of the exercise, in which the exercise may be performed at an intensity of 3.6 MET. Thus, the intensity of the exercise may increase in steps of 0.5 MET, until normal exercise tolerance is achieved by the patient. In some embodiments, if the patient experiences an increase in symptom severity, (e.g., an increase in severity of a symptom of concussion and/or the appearance of one or more new symptoms), the patient may repeat the current stage of the exercise, or revert to a previous stage of exercise intensity.

It is to be understood that a similar regime may be applied to different types of exercises, such as BCTT, running, jogging, walking, isometric exercises, and passive stretching. For example, in some embodiments, a patient may not be in condition to perform exercises requiring movements involved in running, bicycling, etc. The patient may, alternatively, be prescribed one or more of isometric exercises and/or passive stretching exercises. The intensity of isometric and/or stretching exercises may similarly be increased in steps. The increase in MET between steps may be different from different exercises, and vary based on the exercise tolerance of an individual patient.

Accordingly, in various embodiments, GET logic 120 of the local device 105 and/or one or more sensor devices 130b may be configured to prompt the patient 135 to perform one or more of the GET regimes. The GET logic 120 of the local device 105 and/or one or more sensor devices 130b, may further be configured to determine whether the patient 135 has completed a respective GET regime. Once the local device 105 and/or one or more sensor devices 130b has determined that the patient 135 has completed the GET regime, the GET logic 120 may be configured to obtain, via the one or more sensors 130a and/or one or more sensor devices 130b, updated physiologic data from the patient 135.

In some embodiments, the local device 105 may be coupled to the one or more sensor devices 130b. The one or more sensor devices 130b may include a mobile device, such as a smartphone, wearable device, IoT device, a health monitor, or other device configured to obtain physiologic data from a patient 135. Thus, in some embodiments, the local device 105 may be configured to obtain sensor data 125 directly from the patient 135, via the one or more sensors 130a and/or one or more sensor devices 130b. In other embodiments, as previously described, the one or more sensor devices 130b may be configured to transmit sensor data 150 to the remote storage 145. Thus, in some embodiments, the local device 105 may be configured to obtain sensor data 150 from remote storage 145, for example, through communication network 140.

In further embodiments, the local device 105 may be configured to provide sensor data 125 to remote storage 145 and/or the server 160. In turn, the server 160 may be configured to execute GET logic 170 to obtain the physiologic data and to determine a GET regime and/or one or more physiologic targets based on the physiologic data. Thus, in various embodiments, the GET logic 120, 170 may be executed at the local device 105 and/or remotely, for example, at server 160.

Once the one or more physiologic data has been obtained, the local device 105 and/or server 160 may be configured to update a GET regime or obtain an updated GET regime from a clinician. In some embodiments, the local device 105 and/or server 160 may further update the one or more physiologic targets, based on the physiologic data. Historic data may, in turn, be updated based on the newly acquired physiologic data. In one example, if it is determined that HRV is within the HRV target and peak HR is within 90% of the peak HR target, the peak HR target may be increased. The increase, for example, may include an increase of 5 beats per minute (BPM) every 2-3 days. Thus, in one embodiment, HRV may be analyzed relative to the target HRV as the peak HR target is increased. As known to those skilled in the art, improving (e.g., greater) HRV may be a sign of improvement in ANS function. Accordingly, if HRV is within target ranges at a given BPM, a more rigorous GET regime may be performed. In some embodiments, the GET logic 120, 170 may further be configured to modify a GET regime based on a patient's 135 HRV and peak HR, and target HRV and peak HR, and BPV and target BPV, which may be saved, in some examples, as historic data by the GET logic 120, 170.

In further examples, different physiologic measures may be associated with other aspects for brain and heart health. In some embodiments, improvements in other physiologic data, such as BP and HR data, and BPV and HRV data, may correspond to improvement or deterioration with respect to various conditions, including, without limitation, PPCS, postural orthostatic tachycardia syndrome (POTS), fibromyalgia, chronic fatigue syndrome, chronic Epstein-Barr syndrome, chronic lyme syndrome, and other diseases of deconditioning, neurocardiogenic syncope (NCS), Parkinson's disease, and other neurological disorders (including multiple system atrophy (MSA)), hereditary sensory autonomic neuropathies (HSAN), heart valve conditions (e.g., heart valve regurgitation, etc.), heart failure conditions, peripheral vascular disorders, and various neurodegenerative disorders, such as peripheral neuropathy (including diabetic peripheral neuropathy). In some embodiments, the one or more physiologic data may further include data obtained during neurological tests, such as measures of movement and balance, neck range of motion, strength, speed, coordination, memory, attention, speech ability, and subjective feedback. Accordingly, in some embodiments, the GET regime may be designed to improve neurological function and brain and heart health.

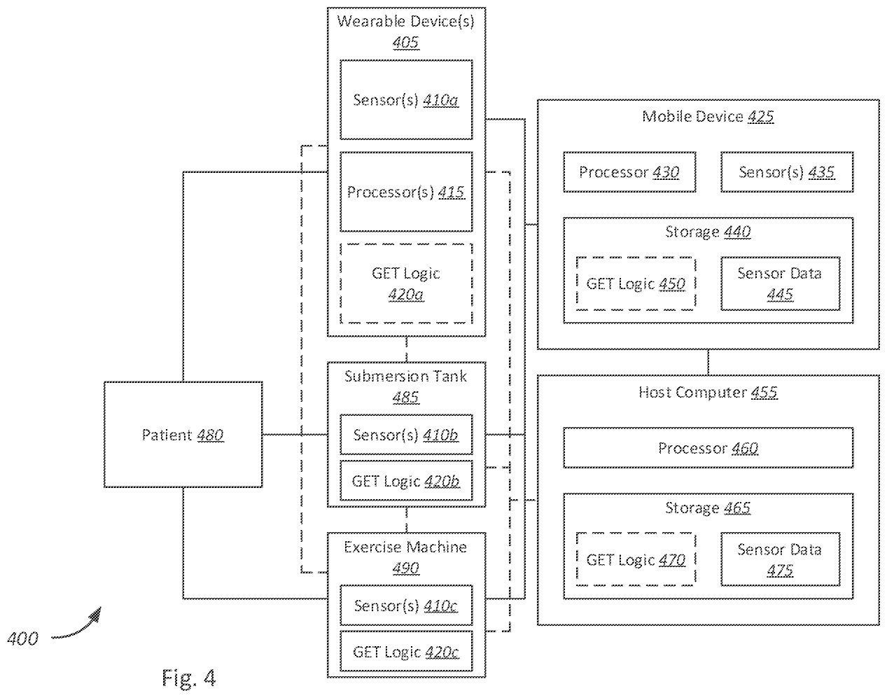

In various embodiments, the GET regime may be determined based on a condition being treated. For example, in some embodiments, a patient 135 may be diagnosed with peripheral neuropathy (e.g., diabetic peripheral neuropathy). Thus, in some embodiments, the GET logic 120, 170 may determine a GET regime specifically to treat diabetic peripheral neuropathy. For example, in response to a determination that the patient 135 is diagnosed with diabetic peripheral neuropathy, a GET regime may include one or more exercise routines performed with the patient's feet submerged underwater. For example, in some embodiments, the feet of the patient 135 may be placed in a submersion tank as described below with respect to FIG. 4. Continuing with the example above, the one or more exercise routines of the GET regime may be performed using the feet, while the feet are submerged in water. In some embodiments, the one or more exercise routines may be performed while the feet remain submerged during each exercise routine. Thus, the submersion tank may be configured to keep the feet of the patient submerged through the range of motion of each of the one or more exercise routines. In some embodiments, the temperature of the water or other submersion medium (also referred to as a liquid medium) may be held at a first temperature while one or more exercise routines are performed. In one example, the water may be held at a temperature of 60 degrees Fahrenheit. In further embodiments, the GET regime may be performed by the patient in a temperature-controlled room, in which the air temperature is maintained at a desired temperature, for example, 60 degrees Fahrenheit. Thus, the entire body of the patient 135 may be cooled by air cooling. In one example, the temperature-controlled room may be a walk-in cooler.

In various embodiments, a GET regime may be determined based on a condition to be treated. For example, GET regime may be determined for a patient 135 to treat a disease of deconditioning, such as POTS, fibromyalgia, chronic fatigue syndrome, chronic Epstein-Barr syndrome, chronic lyme syndrome, or other diseases of deconditioning. The GET regime may, in some embodiments, include one or more exercise routines in which at least the patient's 135 lower body (e.g., a lower half of the patient's 135 body, lower extremities, etc.) is submerged while the one or more exercise routines are performed. For example, in some embodiments, the lower body of the patient 135 may be placed in a submersion tank (such as a pool) as described below with respect to FIG. 4. In some embodiments, the patient 135 may be entirely submerged, with the patient connected to an oxygen supply. In other embodiments, the patient's 135 body may be submerged to varying degrees, including all or part of the upper body of the patient. In yet further embodiments, the patient 135 may be placed in a temperature-controlled room (e.g., a cooled room), such as a walk-in cooler, where the GET regime may be performed by the patient 135 at a cooled ambient temperature. In some embodiments, the GET regime may be performed by the patient 135 in any of the temperature-controlled room (e.g., a cooled room), pool, tub, or other submersion tank, as long as the lower body of the patient is cooled during performance of the GET regime.

The lower body of the patient 135 may include all parts of the body at or below the patient's hips. Thus, the lower body may include, without limitation, the patient's body from the patient's toes to their hips, inclusive of the hips and toes. Thus, the submersion tank may be configured to keep the patient's lower body submerged in the submersion medium throughout an entire range of motion in order to complete the one or more exercise routines. In some embodiments, the submersion tank may include a volume into which the patient 135 may position their lower body and configured to create a seal around the patient's body between the upper and lower body. Thus, while the lower body is submerged in the submersion medium, the upper body of the patient (e.g., above the hips, non-inclusive of the hips) may remain exposed out of the submersion tank. The submersion tank may form a seal around the lower body of the patient, preventing the submersion medium from leaking out of the submersion tank. Thus, in some examples, the submersion tank may, for example, resemble a kayak-like structure configured to accept the lower body of the patient and to form a seal around the lower body of the patient.

In yet further embodiments, a flotation structure may be used in combination with a submersion tank, wherein the flotation structure is configured to suspend or otherwise maintain the patient's upper body out of the submersion medium, while allowing the lower body of the patient to be submerged in the submersion medium. For example, in some embodiments, a catamaran-like flotation structure may be configured to float on a submersion medium in a submersion tank. The submersion tank may, therefore, include a pool or tub in which the flotation structure may be placed. The patient 135, in some embodiments, may be seated or otherwise positioned in, or positioned relative to the flotation structure such that the lower body, or desired part of the patient's body (e.g., feet, legs, etc.) to be selectively submerged in the submersion medium while the upper body of the patient remains out of the submersion medium.

In further embodiments, the one or more exercise routines may be performed at an angle, in which the lower body is positioned above the upper body. Thus, the patient 135 may be positioned at an inclined angle, where the feet are elevated above the head. The patient 135 may be lying down flat in a supine, pronate, or on their sides. For example, the patient 135 may be positioned in the Trendelenburg position while in the submersion tank. Thus, the submersion tank itself may also be placed in an inclined angled position such that the patient may maintain the Trendelenburg position.

In various embodiments, the one or more exercise routines may be performed by the patient utilizing an exercise machine. In some embodiments, the exercise machine may be one or more of a treadmill, step machine, bicycle, stationary bicycle, recumbent bicycle, rowing machine, elliptical, hand cycle, or other suitable equipment. In some embodiments, the exercise machine may for part of the submersion tank. For example, a recumbent bicycle may be placed in the submersion tank within the submersion medium, allowing the patient to perform an exercise routine on the recumbent bicycle while the patient's lower body and/or an appendage, such as the feet, are submerged in a submersion medium. In other embodiments, the exercise machine may be a pedal assembly, as described below with respect to FIG. 4.

In various embodiments, the GET logic 120, 170 may further be configured to diagnose, treat, or both diagnose and treat a condition of the patient. For example, in some embodiments, a GET regime may include performing an exercise in which the patient's 135 feet are fully submerged while one or more exercise routines are performed by the patient 135. For example, the one or more exercise routines may include exercising on a bicycle, such as a stationary bicycle, while the feet are submerged. Physiologic data may be collected from the patient before, during, and after performance of the exercises, such as HR, HRV, BP, BPV, appendage temperature, skin temperature, time to return to a baseline skin temperature, among other physiologic data.

In a further embodiments, physiologic data collected from the patient during a cold pressor test may be used to diagnose concussion as well as other diseases of autonomic dysfunction. For example, the BPV of a patient may be obtained before cooling of a hand of the patient and while the hand of the patient is being cooled and/or is cooled. In other embodiments, other appendages of the patient may be cooled, such as the patient's other hand, or a foot of the patient. For example, a BP of the patient may be measured over a first period of time before the patient's hand or other appendage is cooled. BPV over the first period of time may be determined from the BP signal, thus establishing a baseline BPV signal over time. The patient's hand (or other appendage) may then be cooled, for example, by a submerging the patient appendage in cold water (e.g., ice water), or by a cooling device as described in U.S. patent application Ser. No. 16/702,232 (published as U.S. Patent Publication No. US 2020/0100933), and U.S. patent application Ser. No. 16/832,430, the disclosures of which are incorporated herein by reference in their entireties for all purposes. BPV of the patient may then be obtained. For example, in some embodiments, BP of the patient may be collected over a second period of time while the patient's appendage is being cooled. BPV may then be obtained over the second period of time based on the BP signal. Although the above examples derive BPV based on BP measurements, it is to be understood that in some embodiments, BPV may be measured directly from the patient. In some further embodiments, one or more exercise routines may be performed before, during, and after the patient appendage has been cooled as described above, and BP and/or BPV of the patient obtained while the one or more exercise routines are performed by the patient.

Once BPV of the patient have been obtained, frequency domain components of the BPV signal may be analyzed to determine whether the patient is concussed with resulting autonomic dysfunction, which may be contributing to their post-concussive symptoms, or whether it is evident that other diseases resulting in ANS dysfunction are present. In some embodiments, a fast Fourier transform (FFT) may be performed on the BPV signal to obtain the frequency domain components of the BPV signal. Thus, in some examples, an FFT of the BPV signal over the first period of time (before cooling) may exhibit frequency components in the range of 0.04 to 0.15 Hz, known to be reflective of sympathetic outflow. Thus, the FFT of the BPV signal may include a low frequency component in the range of 0.04-0.15 Hz. The amplitude of the low frequency peak may be compared over the first period of time (e.g., a baseline low frequency peak) to an amplitude of the low frequency peak over the second period of time (e.g., during hand cooling). Furthermore, the amplitude of the lower frequency component may be compared to one or more higher frequency components, for example in the range of 0.15 to 0.40 Hz, known to be reflective of respiratory drive. In some examples, the low frequency component may be a relatively higher amplitude than the one or more higher frequency components over the first period of time (before hand cooling), relative to the difference in amplitude of the low frequency component over the second period of time (during hand cooling) and the one or more higher frequency components over the second period of time. In a healthy individual, BPV over the second period of time (e.g., when patient appendage is cooled) will similarly exhibit a low frequency component and one or more higher frequency components. However, in a concussed individual, or an individual with other disorders of autonomic function, the BPV over the second period of time will show an attenuation or absence of the lower frequency component. Thus, the patient may be diagnosed, based on BPV (including FFT analyses of BPV), for concussion or other disorders of autonomic function.

Thus, in various embodiments, GET logic 120, 170 may be configured to determine whether one or more diseases of deconditioning, concussion (including PPCS), or neurodegenerative disorders are present in the patient based on physiologic data collected before, during, and after performance of the GET regime. Determination of whether one or more diseases of deconditioning are present may include a positive or negative identification of the presence of one or more diseases of deconditioning. Alternatively, a determination of whether one or more diseases of deconditioning are present may include determining a probability that the patient has one or more diseases of deconditioning, or determining probability that the patient has diabetic peripheral neuropathy.

In a further example, in some embodiments, the GET regime may include one or more exercise routines that are performed while the patient's feet are elevated above their heads while the lower body of the patient is submerged in the submersion tank. Accordingly, GET logic 120, 170 may be configured to determine whether peripheral neuropathy is present in the patient 135 based on physiologic data collected before, during, and/or after the one or more exercise routines. As previously described, determination of whether peripheral neuropathy is present may include, for example, a positive or negative identification of peripheral neuropathy, or a probability that the patient has peripheral neuropathy.

In yet further embodiments, the local device 105 may be configured to provide the results of the GET to the patient 135. For example, as previously described, in some examples the local device 105 and/or one or more sensor devices 135 may be coupled to a display device. Thus, the local device 105 and/or one or more sensor devices 135 may be configured to display results of the GET via the display. In some embodiments, GET results may include, without limitation, one or more of real-time physiologic data, physiologic data targets, the current GET regime, historic patient physiologic data, a subsequent GET regime, an indication of whether the patient is improving. In yet further embodiments, GET logic 120, 170 may be configured to propose supplemental therapies based on a patient's 135 progress. The proposed supplemental therapies may include, for example, vestibulo-ocular therapy, physical therapy, cognitive behavioral therapy, meditation therapy, or other therapies as known to those skilled in the art. The specific therapy disclosed may be based, at least in part, on the physiologic data, historical patient data, and collateral data as previously described.

In some embodiments, the one or more physiologic data may be recorded by the local device. In further embodiments, the local device may further be configured to obtain patient feedback, including a patient's subjective feedback regarding the GET regime (e.g., a patient's subjective feelings regarding difficulty or satisfaction with the GET regime), symptom severity, and/or the like. In some cases, established surveys may be used to collect such subjective feedback, such as the Godin Leisure Time Exercise Questionnaire and/or the PCSS. This patient feedback can be collected before, after, during, or independently of the GET regime.

In some embodiments, the GET logic 120, 170 may be configured to enroll and/or manage multiple patients. For example, in some embodiments, each patient may be associated with a respective profile. Each profile may be associated with a respective set of parameters, such as a respective GET regime, respective physiologic data targets, respective historic physiologic data, and respective proposed supplemental therapies. In some embodiments, a profile may further be configured to indicate how the GET should be administered to a respective patient. In one example, a first profile may indicate that a first patient should be administered a GET regime while in a clinician's office under supervision of a clinician. A second profile may indicate that a second patient may perform the GET regime from the patient's home while wearing a wearable monitor or other wearable device. In a further example, data from respective profiles may cumulatively be stored in a database, such as remote storage 145. A respective and the computer system, such as the local device 105, may be configured to apply machine learning techniques to the data to develop algorithms for predicting patient recovery and optimizing the GET regime to accelerate patient improvement.

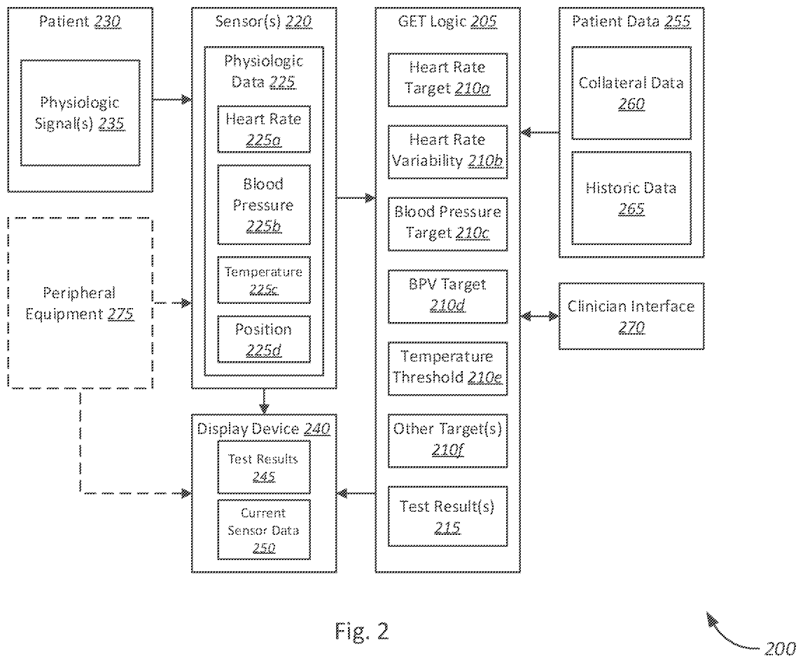

FIG. 2 is a functional block diagram of a graded exercise therapy system 200, in accordance with various embodiments. The system 200 includes GET logic 205, one or more sensors 220, display device 240, patient data 255, and peripheral equipment 275. The one or more sensors 220 may include physiologic data 225, which may further include HR 225a, BP 225b, temperature 225c, and position 225d. GET logic 205 may include one or more HR targets 210a, HRV 210b, BP target 210c, BPV target 210d, temperature threshold 210e, other targets 210f, and one or more test results 215. Patient data 255 may include collateral data 260 and historic data 265. Display device 240 may include test results 245 and current sensor data 250. The system 200 may also include clinician interface 270. A patient 230 may further include one or more physiologic signals 235, which may be measured by the one or more sensors 220. It should be noted that the various components of the system 200 are schematically illustrated in FIG. 2, and that modifications to the system 200 may be possible in accordance with various embodiments.

In various embodiments, the GET logic 205 may be coupled to the one or more sensors 220. The one or more sensors 220 may, in turn, be coupled to the patient 230. The one or more sensors 220 may further be coupled to the display device 240 and peripheral equipment 275. GET logic 205 may further be coupled to the patient data 255 and further be configured to receive a clinician input via the clinician interface 270.

As previously described, the GET logic 205 may include computer readable instructions executable by a processor, such as on a local computer, host computer, server computer, mobile device, remote device, wearable device, or other computer device, to perform one or more of the processes described herein. Accordingly, it is to be understood that GET logic 205, as depicted here, may be implemented across one or devices. Similarly, as previously described, the one or more sensors 220 may include a camera or other image sensor, heart rate monitor, pulse oximeter, gyroscope, accelerometer, wireless transceivers, thermometers or other thermal sensors, microphone, speaker, or acoustic transceiver. The one or more sensors 220 may, like the GET logic 205, be implemented on one or more devices, such as a wearable device, mobile device, and/or a health monitoring device. The one or more sensors 220 may be coupled to the patient 230 to obtain one or more physiologic signals 235 from the patient. Physiologic signals may be signals generated by the patient's 230 body and/or determined from a measurement taken from the patient's body. The one or more sensors 220 may, thus, be configured to generate, as an output of sensor data, the physiologic data 225 from the physiologic signals 235. Physiologic data 225 may include, for example, HR 225a, BP 225b, temperature 225c, and position 225d. Temperature 225c may include, for example, skin temperature taken from a specific part of the patient's body. Position 225d may include a position and/or orientation of a patient's head, neck, or other body part.

Accordingly, in various embodiments, the one or more sensors 220 may further be configured to be coupled to the patient 230 in various ways, depending on the particular sensor and desired physiologic data 225 to be collected from the patient 230. For example, in some embodiments, the sensor 220 may be a biometric monitor, comprising one or more of a pulse oximeter, HR monitor, skin and BP monitor. The biometric monitor may be coupled to the patient in various locations as known to those skilled in the art, such that the biometric monitor remains in contact with the skin of the patient throughout the performance of the GET regime. Suitable body locations may include, without limitation, fingertips, wrist, hand, chest, neck, forehead, ears/earlobe (internally and/or externally), legs, calf, ankles, feet, toes, or other suitable body part. Thus, according to some embodiments, the one or more sensors 220 may be part of a biometric monitor worn around the wrist of the patient 230, the biometric monitor comprising the one or more sensors 220 and configured to capture respective physiologic data.

Accordingly, in various embodiments, the GET logic 205 may be configured to obtain physiologic data 225 from the one or more sensors 220. The GET logic may then process the physiologic data 225 to determine, for example, target physiologic data for one or more GET regimes. For example, target physiologic data may include a HR target 210a, HRV target 210b, BP target 210c, BPV target 210d and one or more other targets 210f. For example, the one or more other targets 210f may include physiologic targets (e.g., a peak HR target, etc.), and/or temperature threshold 210e for respective tests that may be administered to the patient 230. The GET logic 205 may further be configured to determine parameters for evaluating the patient 230 and/or performance of the patient 230. In some embodiments, target physiologic data may be determined based, at least in part, on sensor data collected by the one or more sensors 220. For example, in some embodiments, GET logic 205 may include one or more algorithms configured to determine one or more physiologic targets, and one or more GET routines, based on the physiologic data 225 obtained from the one or more sensors 220.

Accordingly, The GET logic 205 may be configured to determine, for example, HRV 210b and/or one or more other test results 215. For example, the one or more test results 215 may include, in some embodiments, performance on BCTT and BCBT, which may include peak HR, time to return to resting HR. In some embodiments, performance on the ice bucket test and/or appendage cooling test may include physiologic data such as appendage temperature, skin temperature, time to return to baseline skin temperature, etc.

In further embodiments, the GET logic 205 may be configured to determine the one or more other targets 210f, including target physiologic data (e.g., HR target 210a, BP target 210c, HRV target 210b, BPV target 210d), test results 215, and temperature threshold 210e, based, at least in part, on patient data 255. As previously described, patient data 255 may include collateral data 260 and historical data 265. Accordingly, GET logic 205 may further be configured to update one or more physiologic data targets, or a GET routine itself, based on the physiologic data 225. In some further embodiments, the GET logic 205 may further determine one or more physiologic data targets and/or a GET routine further based on the patient data 255 and test results 215.

In yet further embodiments, the GET logic 205 may be configured to determine the one or more other targets 210f, including target physiologic data (e.g., HR target 210a, BP target 210c, HRV target 210b, BPV target 210d), test results 215, and temperature threshold 210e, based, at least in part, on clinician input from the clinician interface 270. Accordingly, in various embodiments, the GET logic 205 may be configured to include a clinician interface, via which input from a clinician may be obtained. For example, clinician input may include physiologic data targets and/or thresholds, or adjustments to physiologic data targets and/or thresholds, provided by a clinician via the clinician interface. Accordingly, adjustments may be provided to for any of the physiologic data targets, such as HR target 210a, HRV targets 210b, BP target 210c, BPV target 210d, temperature threshold 210e, and one or more other targets 210f. In some embodiments, clinician input may be provided by a clinician in real-time or substantially real-time, while in other embodiments, clinician input may be provided by a clinician and stored for later access by the GET logic 205 when GET is provided to the patient 230. In some embodiments, GET logic 205 may be configured to provide real-time physiologic data measurements and/or test results 215 back to the clinician via the clinician interface 270.

In some alternative embodiments, GET logic 205 may further include one or more machine learning (ML) algorithms configured to perform one or more of the processes described above. For example, the ML algorithms may include one or more of neural network algorithms, decision tree algorithms, clustering algorithms, deep learning algorithms, reinforcement learning algorithms, or other suitable machine learning algorithms. The GET logic 205 may, accordingly, in such embodiments, create feature sets (e.g., feature vectors), based at least in part on one or more of the physiologic data 225, patient data 255, clinician input 270, and test results 245. Based on the feature inputs, the GET logic 205 may then determine one or more physiologic data targets, thresholds, and/or test results.

In various embodiments, GET logic 205 may further be configured to determine a GET regime to be performed by the patient 230. The GET regime may, in some embodiments, be obtained from a clinician via the clinician interface 270. In other embodiments, the GET logic 205 may be configured to determine a GET regime based on baseline physiologic data, current physiologic data 220 and patient performance, patient data 255, and/or a predetermined exercise routine. In some embodiments, based on the test result(s) 215 and/or performance of the patient 230 on a current GET regime, the GET regime may be updated by the GET logic 205 to reflect the most current performance of the patient 230 and/or physiologic data 225 of the patient 230. Thus, the GET regime may, in various embodiments, be updated iteratively by GET logic 205, based on the performance of the patient 230 and/or physiologic data 225 from the patient.

In various embodiments, the display device 240 may be coupled to GET logic 205. GET logic 205 may, accordingly, be configured to cause the display device 240 to display test results 245 and/or current sensor data 250 in substantially real-time. In some embodiments, the one or more sensors 220 may be coupled to the display device 240 directly, and thus display current sensor data 250 in substantially real-time. As used herein, substantially real-time refers to real-time data that is displayed in near real-time while accounting for signal propagation and processing delays. In yet further embodiments, the display device 240 may further be configured to display historic data 265 or other patient data 255, clinician input, GET regime instructions (e.g., exercise instructions), and/or provide other feedback to the patient 230.

In further embodiments, the system 200 may further include peripheral equipment 275. Peripheral equipment 275 may include equipment used to administer various tests to the patient 230. For example, peripheral equipment 275 may include one or more of a treadmill, step machine, bicycle, stationary bicycle, recumbent bicycle, rowing machine, elliptical, hand cycle, appendage cooling system, head/neck position tracking system, or other suitable equipment. Accordingly, peripheral equipment 275 may further include one or more sensors 220 or otherwise provide physiologic data 225 to GET logic 205. In yet further embodiments, GET logic 205 may be configured to control peripheral equipment 275 based on the physiologic data 225, physiologic data targets, and/or a GET regime. For example, a GET regime may have an HR target 210a. Thus, in one example, the GET logic 205 may be configured to cause the peripheral equipment 275 (e.g., a treadmill) to be operated at a specific speed and/or incline may be controlled to cause the patient 230 to reach the HR target 210a in a manner desired by the clinician, or as otherwise indicated by the GET regime. In some embodiments, the peripheral equipment 275 may further be coupled to the display device 240, which may display, for example, a speed, incline, position, temperature, or other information regarding the patient 230 or patient 230 performance.

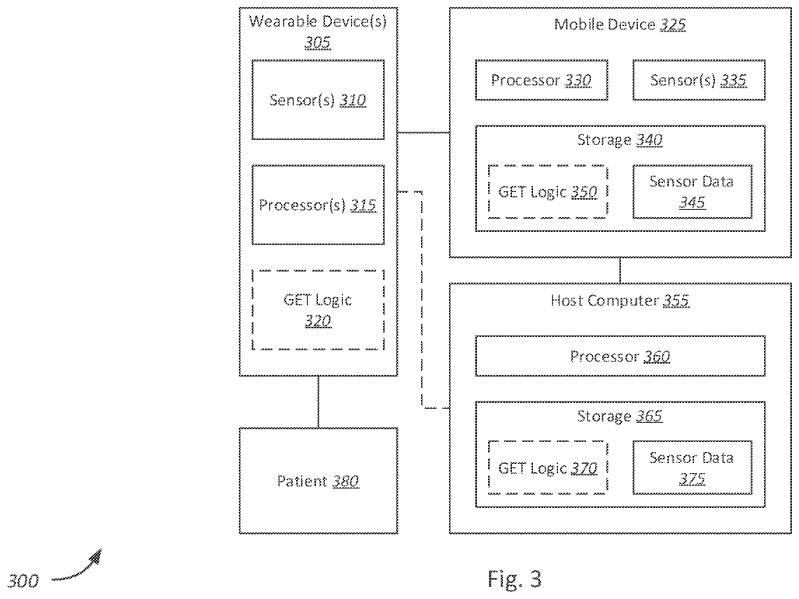

FIG. 3 is a hardware block diagram of a system 300 for providing graded exercise therapy, in accordance with various embodiments. The system 300 includes one or more wearable devices 305, mobile device 325, and host computer 355. The one or more wearable devices 305 may respectively include one or more sensors 310, one or more processors 315, and Get logic 320. The mobile device 325 may similarly include a processor 330, one or more sensors 335, and storage 340. Storage 340 may further include sensor data 345 and GET logic 350. The host computer 355 may include processor 360 and storage 365. The storage 365 may include sensor data 375 and GET logic 370. It should be noted that the architecture and various components of the system 300 are schematically illustrated in FIG. 3, and that modifications to the system 300 may be possible in accordance with various embodiments.

In various embodiments, the one or more wearable devices 305 may be coupled to a patient 380. The one or more wearable devices 305 may further be coupled to one or both of the mobile device 325 and/or host computer 355. The mobile device 325 may be coupled to the one or more wearable devices 305 and host computer 355. Similarly, the host computer 355 may be coupled to one or more of the one or more wearable devices 305 and the mobile device 325.

The one or more wearable devices 305 may comprise, for example, a wearable health monitor, smart watch, or other wearable device. The one or more wearable devices 305 may further include one or more sensors 310 configured to obtain physiologic data from the patient 380. As previously described, the one or more sensors 310 may include, without limitation, a camera or other image sensor, heart rate monitor, pulse oximeter, gyroscope, accelerometer, wireless transceivers, thermometers or other thermal sensors, microphone, speaker, or acoustic transceiver. In some embodiments, the one or more wearable devices 305 may be configured to provide the physiologic data (e.g., sensor data 345, 375) to the mobile device 325 and/or the host computer 355. The mobile device 325 and host computer 355 may, in turn, store the sensor data 345, 375 in respective storage 340, 365.

Similarly, the mobile device 325 may include, for example, a wireless communication device, such as a smartphone, tablet, or laptop computer. The mobile device 325 may similarly include one or more sensors 335, which may, in some embodiments, be configured to obtain physiologic data from the patient 380. In some embodiments, the mobile device 325 may be configured to store sensor data 345 obtained from the one or more wearable devices 305 and/or the one or more sensors 335 in storage 340. Accordingly, in some embodiments, the mobile device 325 may be configured to obtain sensor data 345 from the one or more wearable devices 305. The mobile device 325 may be coupled to the one or more wearable devices 305 wirelessly and/or through a wired connection. Suitable connections may include, without limitation, a Bluetooth, Wi-Fi (e.g., a network connection operating under an 802.11x protocol), infrared, RF, or other wireless transceiver suitable for communication between a wearable device 305 and mobile device 325. The mobile device 325 may further be configured to transmit the sensor data 345 to host computer 355 for further processing by GET logic 370. In other embodiments, the mobile device 325 may include GET logic 350, configured to process the sensor data 345, as previously described.

The host computer 355 may include, for example, a desktop computer, workstation, server computer, remote computer, or other suitable computer device. The host computer 355 may be configured to obtain, from the mobile device 325 and/or one or more wearable devices 305, sensor data 375, and to process the sensor data 375 according to GET logic 370.

Accordingly, GET logic 370 may be executable by the host computer 355 to provide GET to the patient 380. Providing GET to the patient 380 may include controlling one or more of the mobile device 325 and/or one or more wearable devices 305 to guide or otherwise instruct the patient 380 to perform a GET regime. Physiologic data from the patient 380 may be obtained by the various sensors 310, 335 as sensor data 375, which may be processed by the host computer 355. For example, as previously described, the sensor data 375 may be used to determine a new GET regime, change a GET regime, determine one or more physiologic data targets, thresholds, and to evaluate the performance of the patient 380 (e.g., generate test results). The GET logic 370 may, accordingly, be configured to iteratively provide GET, and to update a GET regime, one or more physiologic data targets, thresholds, and to evaluate performance and/or improvement of the patient 380. In yet further embodiments, the GET logic 370 may further be configured to obtain clinician input and other patient data. In further embodiments, one or more of the processes described above may be performed by the mobile device 325 and/or one or more wearable devices 305, which may further respectively include GET logic 320, 350.

In one example, baseline physiologic data may be established for the patient 380. In various embodiments, the patient 380 may be instructed, by the mobile device 325 and/or one or more wearable devices 305, to perform an initial GET regime, which may include an exercise routine. In various embodiments, sensor data 345 may be collected before, during, and after performance of the initial GET regime. Based on the physiologic data collected, baseline physiologic data may be established. Baseline physiologic data may, accordingly, include, without limitation, HR data, HRV, peak HR, BP, BPV, and other physiologic data when GET is started by the patient 380.

In some embodiments, GET logic 320, 350, 370 may be configured to further diagnose a patient and guide therapeutics (e.g., a GET regime or other therapy), based on the physiologic data. As previously described, in some examples, an FFT analysis of patient BPV may be utilized to determine whether the patient is concussed with resulting autonomic dysfunction, which may be contributing to their post-concussive symptoms, or whether it is evident that other diseases resulting in ANS dysfunction are present. In various embodiments, BPV may be obtained from the patient by the one or more sensors 310 of the wearable device 305, or alternatively, derived from BP, which may be measured by one or more sensors 310 from the patient. Thus, in some embodiments, and FFT analysis of BPV may be performed. In some embodiments, a clinician may be able to remotely view and guide GET and/or other therapies based on the FFT analysis of BPV of the patient. In some further embodiments, the GET logic 320, 350, 370 may further be configured to determine one or more exercise routines to be included in the GET regime and/or adjust one or more exercise routines of the GET regime based on the FFT analysis of BPV.

In some embodiments, once baseline physiologic data has been established, the GET logic 320, 350, 370 may be configured to determine, based on the baseline physiologic data, one or more physiologic data targets and thresholds. For example, physiologic data targets may include HR target, peak HR target, HRV target, BP target, BPV target. Thresholds may include performance thresholds on various tests, such as an appendage cooling test. For example, thresholds may include, without limitation, a temperature threshold, a threshold time to return to target temperature, threshold amplitude of the lower frequency component of a BPV signal, or other threshold. In various embodiments, the physiologic data targets and thresholds may reflect an expected iterative improvement in the patient 380. In some embodiments, the physiologic data targets and thresholds may be indicative of the performance of the patient 380 on various GET regimes.

In some embodiments, the one or more physiologic data targets and thresholds may be obtained from a clinician. In further embodiments, the GET logic 320, 350, 370 (e.g., one or more of the wearable devices 305, mobile device 325 and/or hosts computer 355) may be configured to automatically determine the one or more physiologic data targets and thresholds based on the baseline physiologic data. In yet further embodiments, as previously described, GET logic 320, 350, 370 may further include one or more ML algorithms configured to determine physiologic data targets and thresholds based on the baseline physiologic data.

Once the physiologic data targets have been established, the GET regime may be performed at a later time by the patient. For example, in some embodiments, the GET regime may be performed daily, every other day, weekly, or with any other suitable frequency as directed by a clinician or as desired by the patient 380. In other embodiments, the GET regime may include one or more different routines, exercises, and/or tests. Thus, different routines, exercises, and tests may be performed at varying frequencies by the patient 380. In other embodiments, the GET logic 320, 350, 370 may be configured to determine a frequency with which the GET regime should be performed by the patient 380.

In various embodiments, the one or more routines of the GET regime may vary depending on the ability of a patient 380 to perform the one or more routines. For example, in some patients 380, the one or more exercises may vary in intensity and types of movement. For example, if the patient 380 is unable to perform an exercise, such as walking, jogging, running, pedaling, stepping, climbing, isometric exercises etc., the exercise may instead include movements of the patient 380 appendages, such as a hands, feed, toes, or legs. For example, if the patient 380 suffers from peripheral neuropathy, the patient 380 may simply be instructed to wiggle their toes. In further embodiments, the intensity of the exercise may also vary based on an ability of the patient 380 to perform the exercise. Intensity may include physiological data targets, such as a higher HR corresponding to a higher intensity, or the intensity with which an exercise is performed (e.g., a speed of walking, jogging, running, duration, increasing incline, etc.).

In further embodiments, a temperature of a submersion medium may be controlled according to an ability of the patient 380 to perform an exercise or movement. For example, the temperature of the submersion medium may be decreased according to the ability of the patient 380 to perform the movement. In some embodiments, the temperature of the submersion medium may be set lower the less that the patient 380 is able to move their appendages or perform an exercise. As the patient's 380 ability to perform the one or more movements and/or exercises increases, the water temperature may correspondingly be increased. In embodiments where the patient 380 performs the GET regime in a cooled room, the temperature of the cooled room may similarly be modulated according to the ability of the patient 380 to perform the GET regime.

Once the GET regime has been performed, the baseline physiologic data may be replaced with current (e.g., updated) physiologic data. The current physiologic data may be compared, via the GET logic 320, 350, 370, to the one or more physiologic data targets. For example, a newly obtained HR may be compared against a target HR, HRV may be compared against an HRV target, BPV may be compared against a BPV target, etc.

Accordingly, in various embodiments, GET logic 320, 350, 370 may further be configured to update iteratively each of the one or more physiologic data targets, thresholds, and/or the GET regime. For example, if one or more of the physiologic data targets are met, the GET logic 320, 350, 370 may increase a target HR (e.g., by 5 bpm, etc.), increase the target HRV (e.g., the range of variability), and increase an intensity of an exercise regime. Subsequently, the updated GET regime may be performed by the patient 380 at the prescribed time (e.g., based on the frequency as discussed above), and current physiologic data may be used to again iteratively update the one or more physiologic data targets, thresholds, and/or the GET regime. In further embodiments, the GET logic 320, 350, 370 may be configured to further obtain patient data, including historic data and collateral data regarding the patient, and/or clinician input. As previously described, in some embodiments, the GET logic 320, 350, 370 may be configured to determine the one or more physiologic data targets, thresholds, and/or the GET regime based on the patient data. Similarly, clinician input may be used to establish and/or alter one or more physiologic data targets, thresholds, and/or the GET regime.

The GET logic 320, 350, 370 may, accordingly, determine whether a patient 380 is improving as expected based on whether the one or more physiologic data targets or thresholds are met by the patient. If the targets are met, the GET logic 320, 350, 370 may continue to iteratively update the one or more physiologic data targets or thresholds until physiologic data targets and/or thresholds are within a desired range of values (e.g., within a healthy or asymptomatic range, within an expected range of values for a fully recovered individual). In some embodiments, if no improvement is detected, the GET logic 320, 350, 370 may be configured to propose one or more supplemental therapies as previously described. Supplemental therapies may include, without limitation, vestibulo-ocular reflect (VOR) testing, physical therapy, cognitive behavioral therapy, meditation therapy, or other therapies as known to those skilled in the art. In various embodiments, the specific supplemental therapy suggested by the GET logic 320, 350, 370 may be determined based on patient data, one or more physiologic data, clinician input, or by a ML algorithm.

FIG. 4 is a hardware block diagram of an alternative system 400 for providing graded exercise therapy, in accordance with various embodiments. The system 400 includes one or more wearable devices 405, mobile device 425, host computer 455, submersion tank 485, and exercise machine 490. The one or more wearable devices 405 may respectively include one or more sensors 410a, one or more processors 415a, and GET logic 420a. The mobile device 425 may similarly include a processor 430, one or more sensors 435, and storage 440. Storage 440 may further include sensor data 445 and GET logic 450. The host computer 455 may include processor 460 and storage 465. The storage 465 may include sensor data 475 and GET logic 470. The submersion tank 485 may include one or more sensors 410b and GET logic 420b. The exercise machine 490 may include one or more sensors 410c and GET logic 420c. It should be noted that the architecture and various components of the system 400 are schematically illustrated in FIG. 4, and that modifications to the system 400 may be possible in accordance with various embodiments.

In various embodiments, the one or more wearable devices 405 may be coupled to a patient 480. The one or more wearable devices 405 may further be coupled to one or both of the mobile device 425, host computer 455, submersion tank 485, exercise machine 490, and/or the one or more sensors 410b, 410c. The mobile device 425 may be coupled to the one or more wearable devices 405, host computer 455, submersion tank 485, exercise machine 490, and/or the one or more sensors 410b, 410c. Similarly, the host computer 455 may be coupled to one or more of the one or more wearable devices 405, the mobile device 425, submersion tank 485, exercise machine 490, and/or the one or more sensors 410b, 410c.

Like the system 300 of FIG. 3, GET logic 470 may be executable by the host computer 455 to provide GET to the patient 480. In further embodiments, the GET may be provided to the patient via one or more of the mobile device 425, one or more wearable devices 405, submersion tank 485, or exercise machine 490, each of which may execute a respective instance of the GET logic 450, 420a-420c to provide GET to the patient 480.

In various embodiments, providing GET to the patient 480 may include controlling one or more of the mobile device 425, wearable device 405, submersion tank 485, and/or exercise machine 490 to guide or otherwise instruct the patient 380 to perform a GET regime. In various embodiments, the GET regime may include performing a set of one or more exercises via the exercise machine 490. For example, the exercise machine 490 may include, without limitation, a stationary bicycle, recumbent bicycle, treadmill, step machine, rowing machine, elliptical, hand cycle, or other exercise equipment as known to those skilled in the art. In some embodiments, the exercise machine 490 may include at least one pedal assembly comprising a pedal coupled to a crank arm, or other suitable rigid shaft, having a proximal end and a distal end. The pedal may be configured to be rotatable around a distal end of the crank arm. The proximal end of the crank arm may, in turn, be coupled to a sprocket configured to allow the crank arm to rotate about a rotation axis. The sprocket may further be rotatably coupled to body of the exercise machine 490. In some embodiments, the exercise machine 490 may include a second pedal assembly positioned on an opposite side of the sprocket from a first pedal assembly as described above.

In various embodiments, the GET regime may be performed on the exercise machine 490 while the patient 480 and/or exercise machine 490 are at least partially submerged in the submersion tank 485. Accordingly, the submersion tank 485 may include any suitable tank or container within which the exercise machine 490 and patient 480 may be positioned to be at least partially submerged. For example, in some embodiments, the patient 480 may be fully submerged within the submersion tank 485, while in other embodiments, only part of the patient 480 (e.g., an appendage, arms, legs, head, torso, feet, lower body) may be submerged. Accordingly, the submersion tank 485 may include, without limitation, a water tank, barrel, tub, pool, or other suitable container. In some embodiments, the submersion tank 485 may be a water tank configured to seal around the body of the patient. For example, as described above, the water tank may include a volume into which the patient may position their lower body and configured to create a seal around the patient's body between the upper and lower body. Thus, while the lower body is submerged in the submersion medium, the upper body of the patient may remain exposed out of the submersion tank. The submersion tank may form a seal around the lower body of the patient, preventing the submersion medium from leaking out of the submersion tank. Thus, in some examples, the submersion tank may, for example, resemble a kayak-like structure configured to accept the lower body of the patient and to form a seal around the lower body of the patient.

In yet further embodiments, as previously described, a flotation structure may be used in combination with a submersion tank 485. The flotation structure may be configured to suspend or otherwise maintain the patient's upper body out of the submersion medium, while allowing the lower body, or only a desired part (e.g., feet, legs, etc.) of the patient's body to be selectively submerged in the submersion medium. For example, in some embodiments, a catamaran-like flotation structure may be configured to float on a submersion medium in a submersion tank. The submersion tank may, therefore, include a pool or tub in which the flotation structure may be placed. The patient 135, in some embodiments, may be seated or otherwise positioned in, or positioned relative to the flotation structure such that the lower body of the patient is submerged in the submersion medium while the upper body of the patient remains out of the submersion medium.

In various embodiments, the submersion tank 485 may be filled with a desired liquid medium. The liquid medium may include, without limitation, water, such as tap water, salinated water, chlorinated water, or any other treated or untreated water. In some embodiments, the temperature of the liquid medium may be controlled before the GET regime is performed by the patient. Accordingly, in some embodiments, the submersion tank 485 may be configured to control a temperature of the liquid medium before the GET regime has begun. Thus, in some embodiments, the submersion tank 485 may include a cooling device. Cooling devices may include, without limitation, a water cooler (also referred to as a water chiller), such as a pool cooler, or a water heater, heat pump, or other way to control the temperature of the submersion medium. In other embodiments, the liquid medium may be allowed to reach an equilibrium temperature with the ambient environment (e.g., an ambient temperature). In yet further embodiments, the liquid medium may be cooled to a temperature below the ambient temperature, or alternatively below the body temperature of the patient. Conversely, in some embodiments, the liquid medium may be warmed to a temperature above the ambient temperature and/or above the body temperature of the patient.

Thus, in various embodiments, the starting temperature of the liquid medium within the submersion tank 485 may be measured before the GET regime is performed by the patient 480. Similarly, once the patient is placed in the submersion tank 485, physiologic data for the patient 480 may be measured and recorded before the GET regime is performed. In some examples, the patient 480 may be placed within the submersion tank 485 (and liquid medium) for a predetermined acclimation period, the duration of which the patient 480 may first be acclimated to the liquid medium before baseline physiologic data is measured from the patient 480. Physiologic data may include, without limitation, one or more of HR, HRV, peak HR, BP, BPV, blood oxygen saturation (SpO₂), or any other suitable physiologic data.

In some embodiments, a baseline physiologic data may be established for the patient 480. In various embodiments, the patient 480 may be instructed to perform an initial GET regime, which may include an exercise routine. In various embodiments, sensor data 445, 475 may be collected before, during, and after performance of the initial GET regime. Based on the physiologic data collected during the initial GET regime, a baseline physiologic data may be established. Baseline physiologic data may, accordingly, include, without limitation, HR data, HRV, peak HR, BP, BPV, SpO₂, and other physiologic data before, during, and after the initial GET regime is performed by the patient 480. Similarly, a baseline temperature of the liquid medium may be established before, during, and after a GET regime is performed by the patient 480.

In various embodiments, physiologic data gathered by the one or more wearable devices 405, submersion tank 485, and/or exercise machine 490 may be transmitted to one or more of the mobile device 425 or host computer 455. Accordingly, in some embodiments, physiologic data gathered by the one or more sensors 410b of the submersion tank 485 and/or the one or more sensors 410c of the exercise machine 490 may be transmitted to the wearable device 405, for further transmission by the wearable device 405 to a mobile device 425 and/or the host computer 455. In other embodiments, the submersion tank 485 and/or exercise machine 490 may be configured to transmit measured physiologic data to the mobile device 425 and/or host computer 455 directly. Accordingly, in various embodiments, the wearable device 405 may be communicatively coupled to the mobile device 425 and/or host computer 455 and configured to transmit the physiologic data via a wired and/or wireless connection. For example, wired connections may include connections via a wired serial connection (e.g., universal serial bus (USB), etc.), Ethernet, or other twisted pair copper medium, or any other suitable wired interface as known to those skilled in the art. Wireless connections may include, without limitation, a wireless connection utilizing a Bluetooth, Wi-Fi (e.g., a network connection operating under an 802.11x protocol), infrared, RF, or other wireless transceiver suitable for communication between the wearable device 405 and the mobile device 425 and/or host computer 455. Similarly, in some embodiments, the submersion tank 485 and/or exercise machine 490 may include respective wireless transceivers for communication with the wearable device 405, mobile device 425, and/or host computer 455.

Thus, in some embodiments, physiologic data from the patient 480 may be obtained by the various sensors 410a-410c, 435 as sensor data 475, which may be processed by the host computer 455. In some embodiments, sensor data 445, 475 from sensors 410a-410c, 435 may be generated continuously for the duration of the GET regime. Sensor data may include, for example, physiologic data from the patient 480 and/or temperature data, such as temperature of the liquid medium, ambient temperature, body temperature, skin temperature, etc. In some examples, sensor data may be generated continuously for the duration of an exercise (or a timed exercise), monitoring the relevant physiologic data, and in this example, water temperature. As previously described, the sensor data 445, 475 may, therefore, be used to determine a new GET regime, change a GET regime, determine one or more physiologic data targets, thresholds, and to evaluate the performance of the patient 480 (e.g., generate test results). Thus, in some embodiments, one or more of the changes to the GET regime, physiologic data targets, thresholds, and test results may be determined based on one or more of the physiologic data and/or the temperature of the liquid medium (e.g., water temperature). The GET logic 470 may, therefore, be configured to iteratively provide GET, and to update a GET regime, one or more physiologic data targets, thresholds, and to evaluate performance and/or improvement of the patient 480. In yet further embodiments, the GET logic 470 may further be configured to obtain clinician input and other patient data. In further embodiments, one or more of the processes described above may be performed by the mobile device 425 and/or one or more wearable devices 405, which may further include GET logic 420, 450.

For example, once baseline physiologic data has been established, the GET logic 420, 450, 470 may be configured to determine, based on the baseline physiologic data, one or more physiologic data targets and thresholds. For example, physiologic data targets may include HR target, peak HR target, HRV target, BP target, BPV target. Thresholds may include performance thresholds on various tests, such as an appendage cooling test. For example, thresholds may include, without limitation, a temperature threshold, a threshold time to return to target temperature, or other threshold. In various embodiments, the physiologic data targets and thresholds may reflect an expected iterative improvement in the patient 480. In some embodiments, the physiologic data targets and thresholds may be indicative of the performance of the patient 480 on various GET regimes.

In yet further embodiments, physiologic data targets may be determined for one or more temperatures of the liquid medium. Similarly, in some examples, one or more temperatures of the liquid medium may be adjusted for a respective one or more exercises of a GET regime. Thus, in some examples, for a given exercise, one or more physiologic data targets may be set at one or more different starting temperatures of the liquid medium. Furthermore, the starting temperature for one or more exercises of a GET regime may be adjusted. For example, if a patient 480 is able to reach physiologic data targets of a first exercise at a first temperature of the liquid medium, the physiologic data target may be maintained (e.g., unchanged) for the first exercise, but at a second temperature of the liquid medium. In further embodiments, both the physiologic data target and temperature of the liquid medium may be adjusted.

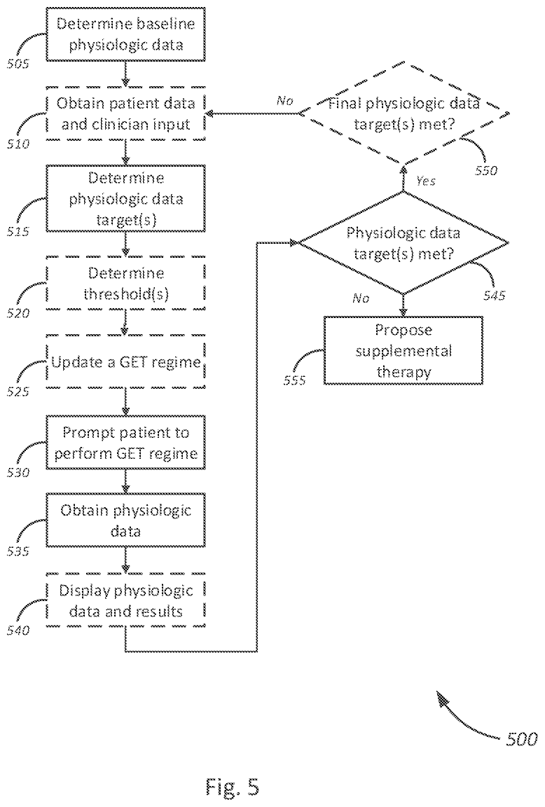

FIG. 5 is a flow diagram of a method 500 for providing graded exercise therapy, in accordance with various embodiments. The method 500 begins, at block 505, by determining, via GET logic, baseline physiologic data. As an initial matter, as previously described, to determine the baseline physiologic data, the method 500 may include initializing a mobile device, one or more wearable device, and/or a host machine. Initializing the mobile device, one or more wearable device, and/or host machine may include obtaining a GET application and/or GET logic from a server, and establishing a connection between the mobile device, one or more wearable device, and/or host machine. Once initialized, GET logic may be configured to generate an initial GET regime. In some embodiments, the initial GET regime may be predetermined. In other embodiments, the GET regime may be received, via clinician interface, from a clinician. In other embodiments, the initial GET regime may be generated based, at least in part, on patient data (e.g., collateral data and/or historic data). The patient may then be prompted to perform the initial GET regime, and physiologic data obtained from the patient. The physiologic data obtained from the patient after the initial GET regime may comprise the baseline physiologic data. As previously described, the GET regime may include one or more exercise routines, tests, or any exercise chosen to be performed by the patient. In further embodiments, the baseline physiologic data may further be associated with a first temperature of a liquid medium in which a patient may at least partially be submerged.

As previously described, baseline physiologic data, such as BPV, may be used to diagnose a patient and guide therapeutics (e.g., the GET regime or other therapy), based on the physiologic data. In some examples, an FFT analysis of patient BPV may be utilized to determine whether the patient is concussed with resulting autonomic dysfunction, which may be contributing to their post-concussive symptoms, or whether it is evident that other diseases resulting in ANS dysfunction are present. Thus, in some embodiments, and FFT analysis of BPV may be performed, and a clinician may be able to remotely view and guide GET and/or other therapies based on the FFT analysis of BPV of the patient.

Accordingly, at block 510, patient data and clinician input may further be obtained. As previously described, with the baseline physiologic data, patient data (including clinical data and/or historic data) may be obtained. In further embodiments, clinician input may be obtained. As previously described, clinician input may include, without limitation, one or more exercise routines to be included in the GET regime and/or one or more exercise routines to be modified, for example, based on the outcome of the FFT analysis of the BPV, or other baseline physiologic data as obtained above. The method 500 continues, at block 515, by determining one or more physiologic data targets. As previously described, physiologic data targets may include an HR target, peak HR target, HRV target, BP target, BPV target, blood oxygenation (SpO₂) target, or any other suitable target for physiologic data. As previously described, in various embodiments, the one or more physiologic data targets may be determined, via the GET logic, based on one or more of a patient's current physiologic data, historical physiologic data, collateral data, and/or clinician input. In further embodiments, physiologic data targets may be determined for one or more temperatures of a liquid medium. Similarly, in some examples, one or more temperatures of the liquid medium may be adjusted for a respective one or more exercises of a GET regime. As previously described, for a given exercise of a GET regime, one or more physiologic data targets may be set at one or more different starting temperatures of the liquid medium.

At block 520, the method 500 may continue by determining one or more thresholds. The one or more thresholds may include, for example, thresholds on various tests, such as an appendage cooling test. For example, thresholds may include, without limitation, a temperature threshold, a threshold time to return to target temperature, or other threshold. Like the one or more physiologic data targets, in various embodiments, the one or more physiologic data targets may be determined, via the GET logic, based on one or more of a patient's current physiologic data, historical physiologic data, collateral data, and/or clinician input.

At block 525, the GET regime may be updated by the GET logic. In some embodiments, one or more of the routines, tests, and/or exercises of the GET regime may be updated, via the GET logic, based on the one or more physiologic data targets or thresholds. In some further embodiments, as previously described, the GET regime may be updated based, at least in part, on patient data, current physiologic data, and/or clinician input. For example, in some embodiments, when it has been determined that a patient is improving (e.g., met the one or more physiologic data targets), the physiologic data targets may be increased. Accordingly, in some embodiments, the GET regime may be updated to include a more strenuous exercise. In some embodiments, one or more of the routines, tests, and/or exercises may be added or removed from the GET regime. In another example, if it is determined that a patient has met the physiologic data targets of a first exercise at a first temperature of the liquid medium, the physiologic data target may be maintained (e.g., unchanged) for the first exercise, but at a second temperature of the liquid medium. Accordingly, in various embodiments, the GET regime may comprise one or more target temperature at which one or more exercises may be performed. Thus, the GET regime may further be updated to reflect changes in target temperatures at which the GET regime is to be performed.

At block 530, the patient may be prompted to perform the GET regime. In some embodiments, the GET regime may include one or more routines, tests, and/or exercises that may be performed according to a respective frequency. Accordingly, the patient may be prompted to perform the respective routine, test, and/or exercise of the GET regime. At block 535, physiologic data may be obtained, via one or more sensors, from the patient. As previously described, one or more sensors may be coupled to the patient. The one or more sensors may include, without limitation, a camera or other image sensor, heart rate monitor, pulse oximeter, gyroscope, accelerometer, wireless transceivers, thermometers or other thermal sensors, microphone, speaker, or acoustic transceiver, or the like. In various embodiments, physiologic data may be recorded before, during, and after the patient has performed the GET regime.

At block 540, the method 500 continues, by displaying physiologic data and/or results of the GET. For example, as previously described, in some embodiments, the GET logic may be configured to cause a display device to display test results (e.g., whether a patient has met a physiologic data target and/or threshold), and/or current sensor data in substantially real-time. In some embodiments, the one or more sensors may be coupled to the display device, which may in turn display the sensor data (physiologic data) from the one or more sensors.

The method 500 continues, at decision block 545, by determining whether the one or more physiologic data targets have been met. For example, in various embodiments, GET logic may be configured to determine whether a patient is improving as expected based on whether the one or more physiologic data targets or thresholds are met by the patient. In some embodiments, if the physiologic data targets are met, the method 500 may continue, at decision block 550 by determining whether final physiologic data targets are met. For example, a final physiologic data target may include, without limitation, physiologic data targets and/or thresholds that are within a desired range of values (e.g., within a healthy or asymptomatic range, within an expected range of values for a fully recovered individual). If it is determined that the physiologic data targets are final physiologic data targets, the method 500 may continue by continuing the GET at the final physiologic data targets, or by termination of the GET. However, if it is determined that final physiologic data targets are not met, the method 500 may continue, at block 510, by obtaining updated patient data and/or clinician input, and continuing to iteratively update the one or more physiologic data targets or thresholds, at blocks 515 and 520, respectively.

In some embodiments, if no improvement is detected, at decision block 545, the method 500 may continue, at block 555, by proposing a supplemental therapy. In various embodiments, as previously described, GET logic may be configured to propose one or more supplemental therapies as previously described. Supplemental therapies may include, without limitation, VOR testing, physical therapy, cognitive behavioral therapy, meditation therapy, or other therapies as known to those skilled in the art. In various embodiments, the specific supplemental therapy suggested by the GET logic may be determined based on patient data, one or more physiologic data, clinician input, or by a ML algorithm.

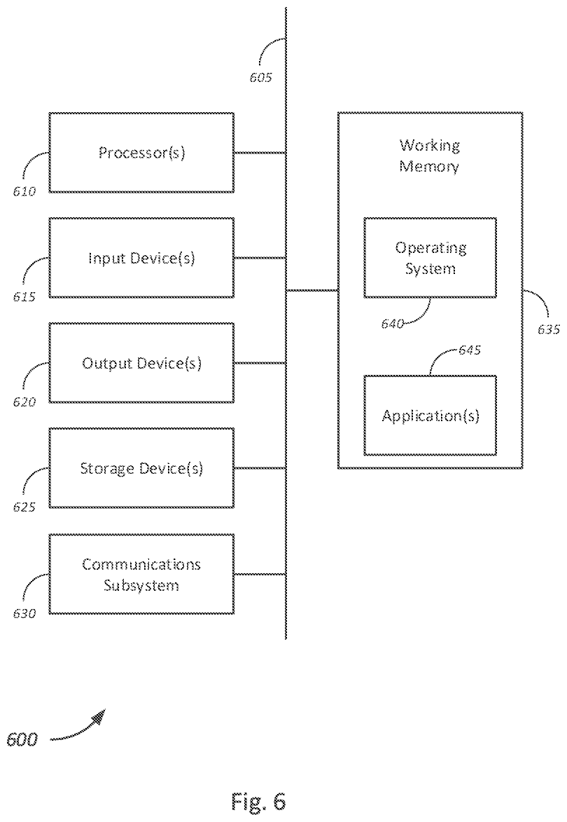

FIG. 6 is a schematic block diagram of a computer system 600 for providing graded exercise therapy, in accordance with various embodiments. The computer system 600 is a schematic illustration of a computer system (physical and/or virtual), such as a user device or server, which may perform the methods provided by various other embodiments, as described herein. It should be noted that FIG. 6 only provides a generalized illustration of various components, of which one or more of each may be utilized as appropriate. FIG. 6, therefore, broadly illustrates how individual system elements may be implemented in a relatively separated or relatively more integrated manner.

The computer system 600 includes multiple hardware (or virtualized) elements that may be electrically coupled via a bus 605 (or may otherwise be in communication, as appropriate). The hardware elements may include one or more processors 610, including, without limitation, one or more general-purpose processors and/or one or more special-purpose processors (such as microprocessors, digital signal processing chips, graphics acceleration processors, and microcontrollers); one or more input devices 615, which include, without limitation, a mouse, a keyboard, one or more sensors, and/or the like; and one or more output devices 620, which can include, without limitation, a display device, and/or the like.

The computer system 600 may further include (and/or be in communication with) one or more storage devices 625, which can comprise, without limitation, local and/or network accessible storage, and/or can include, without limitation, a disk drive, a drive array, an optical storage device, solid-state storage device such as a random-access memory (“RAM”) and/or a read-only memory (“ROM”), which can be programmable, flash-updateable, and/or the like. Such storage devices may be configured to implement any appropriate data stores, including, without limitation, various file systems, database structures, and/or the like.

The computer system 600 may also include a communications subsystem 630, which may include, without limitation, a modem, a network card (wireless or wired), an IR communication device, a wireless communication device and/or chip set (such as a Bluetooth™ device, an 802.11 device, a WiFi device, a WiMax device, a WWAN device, a low-power (LP) wireless device, a Z-Wave device, a ZigBee device, cellular communication facilities, etc.). The communications subsystem 630 may permit data to be exchanged with a network (such as the network described below, to name one example), with other computer or hardware systems, between data centers or different cloud platforms, and/or with any other devices described herein. In many embodiments, the computer system 600 further comprises a working memory 635, which can include a RAM or ROM device, as described above.

The computer system 600 also may comprise software elements, shown as being currently located within the working memory 635, including an operating system 640, device drivers, executable libraries, and/or other code, such as one or more application programs 645, which may comprise computer programs provided by various embodiments, and/or may be designed to implement methods, and/or configure systems, provided by other embodiments, as described herein. Merely by way of example, one or more procedures described with respect to the method(s) discussed above may be implemented as code and/or instructions executable by a computer (and/or a processor within a computer); in an aspect, then, such code and/or instructions can be used to configure and/or adapt a general purpose computer (or other device) to perform one or more operations in accordance with the described methods.

A set of these instructions and/or code may be encoded and/or stored on a non-transitory computer readable storage medium, such as the storage device(s) 625 described above. In some cases, the storage medium may be incorporated within a computer system, such as the system 600. In other embodiments, the storage medium may be separate from a computer system (i.e., a removable medium, such as a compact disc, etc.), and/or provided in an installation package, such that the storage medium can be used to program, configure, and/or adapt a general purpose computer with the instructions/code stored thereon. These instructions may take the form of executable code, which is executable by the computer system 600 and/or may take the form of source and/or installable code, which, upon compilation and/or installation on the computer system 600 (e.g., using any of a variety of generally available compilers, installation programs, compression/decompression utilities, etc.) then takes the form of executable code.

It will be apparent to those skilled in the art that substantial variations may be made in accordance with specific requirements. For example, customized hardware (such as programmable logic controllers, single board computers, FPGAs, ASICs, and SoCs) may also be used, and/or particular elements may be implemented in hardware, software (including portable software, such as applets, etc.), or both. Further, connection to other computing devices such as network input/output devices may be employed.

As mentioned above, in one aspect, some embodiments may employ a computer or hardware system (such as the computer system 600) to perform methods in accordance with various embodiments of the invention. According to a set of embodiments, some or all of the procedures of such methods are performed by the computer system 600 in response to processor 610 executing one or more sequences of one or more instructions (which may be incorporated into the operating system 640 and/or other code, such as an application program 645 or firmware) contained in the working memory 635. Such instructions may be read into the working memory 635 from another computer readable medium, such as one or more of the storage device(s) 625. Merely by way of example, execution of the sequences of instructions contained in the working memory 635 may cause the processor(s) 610 to perform one or more procedures of the methods described herein.

The terms “machine readable medium” and “computer readable medium,” as used herein, refer to any medium that participates in providing data that causes a machine to operate in a specific fashion. In an embodiment implemented using the computer system 600, various computer readable media may be involved in providing instructions/code to processor(s) 610 for execution and/or may be used to store and/or carry such instructions/code (e.g., as signals). In many implementations, a computer readable medium is a non-transitory, physical, and/or tangible storage medium. In some embodiments, a computer readable medium may take many forms, including, but not limited to, non-volatile media, volatile media, or the like. Non-volatile media includes, for example, optical and/or magnetic disks, such as the storage device(s) 625. Volatile media includes, without limitation, dynamic memory, such as the working memory 635. In some alternative embodiments, a computer readable medium may take the form of transmission media, which includes, without limitation, coaxial cables, copper wire and fiber optics, including the wires that comprise the bus 605, as well as the various components of the communication subsystem 630 (and/or the media by which the communications subsystem 630 provides communication with other devices). In an alternative set of embodiments, transmission media can also take the form of waves (including, without limitation, radio, acoustic, and/or light waves, such as those generated during radio-wave and infra-red data communications).

Common forms of physical and/or tangible computer readable media include, for example, a floppy disk, a flexible disk, a hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a computer can read instructions and/or code.

Various forms of computer readable media may be involved in carrying one or more sequences of one or more instructions to the processor(s) 610 for execution. Merely by way of example, the instructions may initially be carried on a magnetic disk and/or optical disc of a remote computer. A remote computer may load the instructions into its dynamic memory and send the instructions as signals over a transmission medium to be received and/or executed by the computer system 600. These signals, which may be in the form of electromagnetic signals, acoustic signals, optical signals, and/or the like, are all examples of carrier waves on which instructions can be encoded, in accordance with various embodiments of the invention.

The communications subsystem 630 (and/or components thereof) generally receives the signals, and the bus 605 then may carry the signals (and/or the data, instructions, etc. carried by the signals) to the working memory 635, from which the processor(s) 610 retrieves and executes the instructions. The instructions received by the working memory 635 may optionally be stored on a storage device 625 either before or after execution by the processor(s) 610.

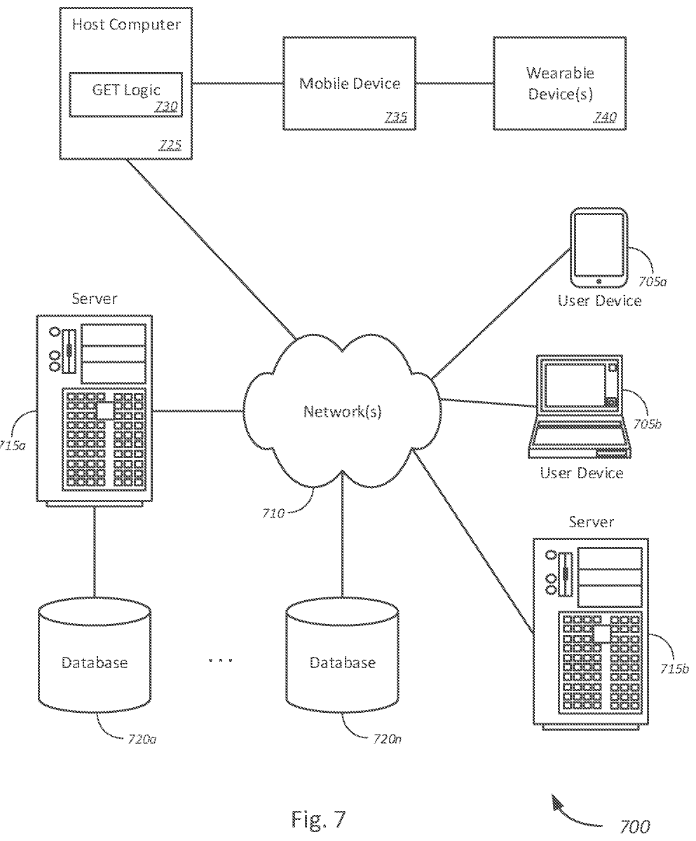

FIG. 7 is a schematic block diagram illustrating system of networked computer devices, in accordance with various embodiments. The system 700 may include one or more user devices 705. A user device 705 may include, merely by way of example, desktop computers, single-board computers, tablet computers, laptop computers, handheld computers, edge devices, wearable devices, and the like, running an appropriate operating system. User devices 705 may further include external devices, remote devices, servers, and/or workstation computers running any of a variety of operating systems. A user device 705 may also have any of a variety of applications, including one or more applications configured to perform methods provided by various embodiments, as well as one or more office applications, database client and/or server applications, and/or web browser applications. Alternatively, a user device 705 may include any other electronic device, such as a thin-client computer, Internet-enabled mobile telephone, and/or personal digital assistant, capable of communicating via a network (e.g., the network(s) 710 described below) and/or of displaying and navigating web pages or other types of electronic documents. Although the exemplary system 700 is shown with two user devices 705a-705b, any number of user devices 705 may be supported.

Certain embodiments operate in a networked environment, which can include a network(s) 710. The network(s) 710 can be any type of network familiar to those skilled in the art that can support data communications, such as an access network, core network, or cloud network, and use any of a variety of commercially-available (and/or free or proprietary) protocols, including, without limitation, MQTT, CoAP, AMQP, STOMP, DDS, SCADA, XMPP, custom middleware agents, Modbus, BACnet, NCTIP, Bluetooth, Zigbee/Z-wave, TCP/IP, SNA™, IPX™, and the like. Merely by way of example, the network(s) 710 can each include a local area network (“LAN”), including, without limitation, a fiber network, an Ethernet network, a Token-Ring™ network and/or the like; a wide-area network (“WAN”); a wireless wide area network (“WWAN”); a virtual network, such as a virtual private network (“VPN”); the Internet; an intranet; an extranet; a public switched telephone network (“PSTN”); an infra-red network; a wireless network, including, without limitation, a network operating under any of the IEEE 802.11 suite of protocols, the Bluetooth™ protocol known in the art, and/or any other wireless protocol; and/or any combination of these and/or other networks. In a particular embodiment, the network may include an access network of the service provider (e.g., an Internet service provider (“ISP”)). In another embodiment, the network may include a core network of the service provider, backbone network, cloud network, management network, and/or the Internet.

Embodiments can also include one or more server computers 715. Each of the server computers 715 may be configured with an operating system, including, without limitation, any of those discussed above, as well as any commercially (or freely) available server operating systems. Each of the servers 715 may also be running one or more applications, which can be configured to provide services to one or more clients 705 and/or other servers 715.

Merely by way of example, one of the servers 715 may be a data server, a web server, orchestration server, authentication server (e.g., TACACS, RADIUS, etc.), cloud computing device(s), or the like, as described above. The data server may include (or be in communication with) a web server, which can be used, merely by way of example, to process requests for web pages or other electronic documents from user computers 705. The web server can also run a variety of server applications, including HTTP servers, FTP servers, CGI servers, database servers, Java servers, and the like. In some embodiments of the invention, the web server may be configured to serve web pages that can be operated within a web browser on one or more of the user computers 705 to perform methods of the invention.

The server computers 715, in some embodiments, may include one or more application servers, which can be configured with one or more applications, programs, web-based services, or other network resources accessible by a client. Merely by way of example, the server(s) 715 can be one or more general purpose computers capable of executing programs or scripts in response to the user computers 705 and/or other servers 715, including, without limitation, web applications (which may, in some cases, be configured to perform methods provided by various embodiments). Merely by way of example, a web application can be implemented as one or more scripts or programs written in any suitable programming language, such as Java™, C, C#™ or C++, and/or any scripting language, such as Perl, Python, or TCL, as well as combinations of any programming and/or scripting languages. The application server(s) can also include database servers, including, without limitation, those commercially available from Oracle™, Microsoft™, Sybase™, IBM™, and the like, which can process requests from clients (including, depending on the configuration, dedicated database clients, API clients, web browsers, etc.) running on a user computer, user device, or customer device 705 and/or another server 715.

In accordance with further embodiments, one or more servers 715 can function as a file server and/or can include one or more of the files (e.g., application code, data files, etc.) necessary to implement various disclosed methods, incorporated by an application running on a user computer 705 and/or another server 715. Alternatively, as those skilled in the art will appreciate, a file server can include all necessary files, allowing such an application to be invoked remotely by a user computer, user device, or customer device 705 and/or server 715.

It should be noted that the functions described with respect to various servers herein (e.g., application server, database server, web server, file server, etc.) can be performed by a single server and/or a plurality of specialized servers, depending on implementation-specific needs and parameters.

In certain embodiments, the system can include one or more databases 720a-720n (collectively, “databases 720”). The location of each of the databases 720 is discretionary: merely by way of example, a database 720a may reside on a storage medium local to (and/or resident in) a server 715a (or alternatively, user device 705). Alternatively, a database 720n can be remote so long as it can be in communication (e.g., via the network 710) with one or more of these. In a particular set of embodiments, a database 720 can reside in a storage-area network (“SAN”) familiar to those skilled in the art. In one set of embodiments, the database 720 may be a relational database configured to host one or more data lakes collected from various data sources. The databases 720 may include SQL, no-SQL, and/or hybrid databases, as known to those in the art. The database may be controlled and/or maintained by a database server.

The system 700 may further include a host computer 725, GET logic 730, mobile device 735, and one or more wearable devices 740. In various embodiments, the host computer 725 may be coupled to the network 710, and mobile device 735. The host computer may further include GET logic 730. As previously described, in various embodiments, the one or more wearable devices 740 may be coupled to a patient, and configured to obtain physiologic data from the patient. Similarly, the mobile device 735 may further include one or more sensors, and be configured to obtain physiologic data from the patient. In various embodiments, the one or more wearable devices 740 and mobile device 735 may be configured to provide physiologic data to the host computer 725. The host computer 725 may be configured to execute GET logic 730, which may include one or more instructions for providing GET to the patient. In various embodiments, the GET logic 730 may be configured to determine one or more physiologic data targets based on the physiologic data obtained from the patient. In some further embodiments, the physiologic data targets may further be based on patient data, such as historical physiologic data, and collateral data regarding the patient. The GET logic 730 may further be configured to instruct the patient, via the mobile device 735 and/or one or more wearable devices 740, to perform a GET regime, including one or more routines, tests, and/or exercises. Physiologic data from the patient may be collected during the GET regime via the one or more wearable devices 740 and/or mobile device 735. The GET logic 730 may be configured to determine whether the physiologic data obtained during the GET regime meets or falls within the physiologic data targets. In further embodiments, one or more thresholds may further be determined by the GET logic 730. The accordingly, GET logic 730 may further determine whether a patient's performance of the GET regime falls within the thresholds determined. In various embodiments, if the physiologic data targets and/or thresholds are met, the GET logic 730 may be configured to iteratively update the physiologic data targets and/or thresholds, according to a patient's progress. In further embodiments, one or more routines, tests, and/or exercises of the GET regime may be updated according to the patient's performance and physiologic data during gathered during the GET regime.

While certain features and aspects have been described with respect to exemplary embodiments, one skilled in the art will recognize that numerous modifications are possible. For example, the methods and processes described herein may be implemented using hardware components, software components, and/or any combination thereof. Further, while various methods and processes described herein may be described with respect to certain structural and/or functional components for ease of description, methods provided by various embodiments are not limited to any single structural and/or functional architecture but instead can be implemented on any suitable hardware, firmware and/or software configuration. Similarly, while certain functionality is ascribed to certain system components, unless the context dictates otherwise, this functionality can be distributed among various other system components in accordance with the several embodiments.

Moreover, while the procedures of the methods and processes described herein are described in sequentially for ease of description, unless the context dictates otherwise, various procedures may be reordered, added, and/or omitted in accordance with various embodiments. Moreover, the procedures described with respect to one method or process may be incorporated within other described methods or processes; likewise, system components described according to a specific structural architecture and/or with respect to one system may be organized in alternative structural architectures and/or incorporated within other described systems. Hence, while various embodiments are described with—or without—certain features for ease of description and to illustrate exemplary aspects of those embodiments, the various components and/or features described herein with respect to one embodiment can be substituted, added and/or subtracted from among other described embodiments, unless the context dictates otherwise. Consequently, although several exemplary embodiments are described above, it will be appreciated that the invention is intended to cover all modifications and equivalents within the scope of the following claims.

What is claimed is:

1. A system comprising:
one or more sensors coupled to a patient;
a host machine coupled to the one or more sensors, the host machine further comprising:
a processor; and
a computer readable medium in communication with the processor, the computer readable medium having encoded thereon a set of instructions executable by the processor to:
determine, via the one or more sensors, a baseline set of physiologic data of the patient;
establish a graded exercise therapy regime for the patient, the graded exercise therapy regime including one or more exercises;
determine one or more physiologic data targets for the patient based, at least in part, on the baseline physiologic data of the patient;
obtain, via the one or more sensors, a first set of physiologic data of the patient during the graded exercise therapy regime;
determine whether the first set of physiologic data meets the one or more physiologic data targets; and
in response to determining that the first set of physiologic data meets the one or more physiologic data targets, update the one or more physiologic data targets based, at least in part, on the first set of physiologic data.

2. The system of claim 1, wherein the set of instructions are further executable by the processor to:
obtain patient data associated with the patient, wherein patient data includes one or more of historical physiologic data and collateral data;
wherein the one or more physiologic data targets for the patient are further based, at least in part, on the patient data, and
wherein collateral data includes at least one of a test result, a patient response to an evaluation, and medical history.

3. The system of claim 1, wherein the one or more physiologic data targets includes target heartrate variability.

4. The system of claim 1, wherein the one or more physiologic data targets includes beat-to-beat blood pressure variability.

5. The system of claim 1, wherein the one or more sensors includes at least one of a heart rate monitor, pulse oximeter, or blood pressure monitor.

6. The system of claim 1, wherein the set of instructions are further executable by the processor to:
obtain, via the host computer, a clinician input, wherein the clinician input is indicative of at least one change to the one or more physiologic data targets; and
update, via the host computer, the one or more physiologic data targets based, at least in part, on the clinician input.

7. The system of claim 1, wherein the set of instructions are further executable by the processor to:
repeat the graded exercise therapy regime until a current set of physiologic data obtained from the patient during the graded exercise therapy regime reaches a final physiologic data target, wherein the final physiologic data target is associated with the absence of autonomic nervous system dysfunction.

8. The system of claim 1, wherein the set of instructions are further executable by the processor to:
propose, in response to determining that the first set of physiologic data does not meet the one or more physiologic data targets, a supplemental therapy for the patient, wherein the supplemental therapy includes at least one of vestibulo-ocular reflex testing, physical therapy, cognitive behavioral therapy, or meditation therapy.

9. The system of claim 1 further comprising a submersion tank configured to store a liquid medium and allow at least part of the patient to be submerged, wherein the set of instructions are further executable by the processor to:
obtain, via the one or more sensors, a first temperature of the liquid medium at at least one of before, during, or after the graded exercise therapy regime has been performed by the patient;
wherein the first set of physiologic data is associated with the first temperature, wherein the update of the one or more physiologic data targets is based, at least in part, on the first temperature.

10. An apparatus comprising:
a processor; and
a computer readable medium in communication with the processor, the computer readable medium having encoded thereon a set of instructions executable by the processor to:
determine, via one or more sensors, a baseline set of physiologic data of a patient;
establish a graded exercise therapy regime for the patient, the graded exercise therapy regime including one or more exercises;
determine one or more physiologic data targets for the patient based, at least in part, on the baseline physiologic data of the patient;
obtain, via the one or more sensors, a first set of physiologic data of the patient during the graded exercise therapy regime;
determine whether the first set of physiologic data meets the one or more physiologic data targets; and
in response to determining that the first set of physiologic data meets the one or more physiologic data targets, update the one or more physiologic data targets based, at least in part, on the first set of physiologic data.

11. The apparatus of claim 10, wherein the set of instructions are further executable by the processor to:
obtain patient data associated with the patient, wherein patient data includes one or more of historical physiologic data and collateral data;
obtain, via the host computer, a clinician input, wherein the clinician input is indicative of at least one change to the one or more physiologic data targets;
update, via the host computer, the one or more physiologic data targets based, at least in part, on the clinician input;
wherein the one or more physiologic data targets for the patient are further based, at least in part, on the patient data, and
wherein collateral data includes at least one of a test result, a patient response to an evaluation, and medical history.

12. The apparatus of claim 10, wherein the one or more physiologic data targets includes target heartrate variability.

13. The apparatus of claim 10, wherein the one or more physiologic data targets includes beat-to-beat blood pressure variability.

14. The apparatus of claim 10, wherein the set of instructions are further executable by the processor to:
repeat the graded exercise therapy regime until a current set of physiologic data obtained from the patient during the graded exercise therapy regime reaches a final physiologic data target, wherein the final physiologic data target is associated with the absence of autonomic nervous system dysfunction.

15. The apparatus of claim 10, wherein the set of instructions are further executable by the processor to:
propose, in response to determining that the first set of physiologic data does not meet the one or more physiologic data targets, a supplemental therapy for the patient, wherein the supplemental therapy includes at least one of vestibulo-ocular reflex testing, physical therapy, cognitive behavioral therapy, or meditation therapy.

16. The apparatus of claim 10, wherein the set of instructions are further executable by the processor to:
obtain, via the one or more sensors, a first temperature of a liquid medium of a submersion tank at at least one of before, during, or after the graded exercise therapy regime has been performed by the patient, wherein at least part of the patient is submerged within the liquid medium of the submersion tank;
wherein the first set of physiologic data is associated with the first temperature, wherein the update of the one or more physiologic data targets is based, at least in part, on the first temperature.

17. A method comprising:
determining, via one or more sensors, a baseline set of physiologic data of the patient;
establishing, via a host computer, a graded exercise therapy regime for the patient, the graded exercise therapy regime including one or more exercises;
determining, via the host computer, one or more physiologic data targets for the patient based, at least in part, on the baseline physiologic data of the patient;
obtaining, via the one or more sensors, a first set of physiologic data of the patient during the graded exercise therapy regime;
determining, via the host computer, whether the first set of physiologic data meets the one or more physiologic data targets; and
in response to determining that the first set of physiologic data meets the one or more physiologic data targets, updating, via the host computer, the one or more physiologic data targets based, at least in part, on the first set of physiologic data.

18. The method of claim 17, further comprising:
obtaining, via the host computer, patient data associated with the patient, wherein patient data includes one or more of historical physiologic data and collateral data;
obtaining, via the host computer, a clinician input, wherein the clinician input is indicative of at least one change to the one or more physiologic data targets;
updating, via the host computer, the one or more physiologic data targets based, at least in part, on the clinician input;
wherein the one or more physiologic data targets for the patient are further based, at least in part, on the patient data, and
wherein collateral data includes at least one of a test result, a patient response to an evaluation, and medical history.

19. The method of claim 17, further comprising:
repeating, via the host computer, the graded exercise therapy regime until a current set of physiologic data obtained from the patient during the graded exercise therapy regime reaches a final physiologic data target, wherein the final physiologic data target is associated with the absence of autonomic nervous system dysfunction; and
proposing, via the host computer, in response to determining that the first set of physiologic data does not meets the one or more physiologic data targets, a supplemental therapy for the patient, wherein the supplemental therapy includes at least one of vestibulo-ocular reflex testing, physical therapy, cognitive behavioral therapy, or meditation therapy.

20. The method of claim 17, further comprising:
obtaining, via the one or more sensors, a first temperature of a liquid medium of a submersion tank at at least one of before, during, or after the graded exercise therapy regime has been performed by the patient, wherein at least part of the patient is submerged within the liquid medium of the submersion tank;
wherein the first set of physiologic data is associated with the first temperature, wherein the update of the one or more physiologic data targets is based, at least in part, on the first temperature.
